(19) United States
(12) Patent Application Publication
(10) Pub. No.: US 2023/0398177 A1
(43) Pub. Date: Dec. 14, 2023

(54) COMPOSITIONS OF ANTI-VIRAL PEPTIDES AND/OR COMPOUNDS AND METHODS OF USE THEREOF

(71) Applicant: The University of Hong Kong, Hong Kong (CN)

(72) Inventors: Hanjun Zhao, Hong Kong (CN); Kwok-Yung Yuen, Hong Kong (CN)

(21) Appl. No.: 18/249,926

(22) PCT Filed: Oct. 22, 2021
(86) PCT No.: PCT/CN2021/125649

§ 371 Date: Apr. 20, 2023

Related U.S. Application Data
(60) Provisional application No. 63/104,312, filed on Oct. 22, 2020.

(51) Int. Cl. A61K 38/17; A61P 31/14; A61K 31/4706; A61K 31/404

(52) U.S. Cl. CPC A61K 38/1729; A61P 31/14; A61K 31/4706; A61K 31/404

(57) ABSTRACT

Broad spectrum antiviral peptides and compositions including therapeutically effective amounts of the antiviral peptides along with a pharmaceutically acceptable carrier are provided. The antiviral compositions show a strong broad spectrum antiviral effect, without resulting to viral resistance. The antiviral compositions are useful for treatment of diseases caused by viral infections, particularly respiratory viruses such as enveloped coronaviruses (SARS-CoV-2, SARS-CoV and MERS-CoV), the pandemic A (H1N1) pdm09 virus, avian influenza A (H7N9) virus, and the non-enveloped rhinovirus.

### FIELD OF THE INVENTION

The invention is generally directed to broad spectrum antiviral peptides and small molecules, and methods of treating antiviral infections.

### BACKGROUND OF THE INVENTION

Novel respiratory viruses often cause severe respiratory tract infections and spread quickly due to the lack of pre-existing immunity. In the recent two decades, three highly pathogenic coronaviruses have crossed species barrier and caused human diseases, including the bat-related severe acute respiratory syndrome (SARS) coronavirus (CoV) (SARS-CoV) in 2003 (Woo et al., Lancet 363, 841-845 (2004), Lau et al., Proc Natl Acad Sci USA 102, 14040-14045 (2005)), the Middle East respiratory syndrome coronavirus (MERS-CoV) since 2012 (Chan et al., Clinical microbiology reviews 28, 465-522 (2015), Yeung et al., Nat Microbiol 1, 16004 (2016)) and the recent 2019 new coronavirus (SARS-CoV-2)(Chan et al., Lancet 395, 514-523 (2020)). Furthermore, the 2009 pandemic influenza A(H1N1)pdm09 virus had led to the 1 st influenza pandemic in the 21 st century, and the avian influenza virus A(H7N9) had caused a large zoonotic outbreak in mainland China (To et al., Lancet Infect Dis 13, 809-821 (2013), Cheng et al., Clinical microbiology reviews 25, 223-263 (2012)). Due to the lack of effective antivirals, especially for coronaviruses, these respiratory viruses are associated with significant morbidity and mortality. Furthermore, these emerging respiratory viruses have also caused severe economic and social disturbances.

The COVID-2019 outbreak has clearly illustrated the importance of broad-spectrum antivirals. While an outbreak of unusual pneumonia was reported in December 2019, the identity of SARS-CoV-2 was reported on Jan. 8, 2020 by China CDC (Li et al., N Engl J Med (2020)). There is not yet a reliable antiviral or vaccine available for therapy or prevention of SARS-CoV-2 infection. Studies showed that the SARS-CoV-2 infected patients may have decreasing level of antibodies (Ibarrondo, et al., N Engl J Med 383, 1085-1087 (2020), Guo, et al., Front Immunol 11, 1936 (2020), Long, et al., Nat Med 26, 1200-1204 (2020), Liu, et al., Clin Microbiol Infect (2020), Sariol, & Perlman, Immunity 53, 248-263 (2020)), which suggested that SARS-CoV-2 vaccine may also have varying duration of protection among different individuals. Furthermore, reports of re-infection hinted that the immune responses to SARS-CoV-2 might not sufficiently protect some patients from re-infection of SARS-CoV-2 (To, et al., Clin Infect Dis (2020)). The antibody-dependent enhancement is another potential side effect of SARS-CoV-2 vaccines (Lee, et al., Nat Microbiol (2020), Arvin, et al., Nature 584, 353-363 (2020)). Broad-spectrum antivirals, not relying on host immune responses against viruses, are urgently needed for treating COVED-19 and other coronavirus infections. Thus, broad spectrum antiviral peptides against SARS-CoV-2 (Xia, et al., Cell Res 30, 343-355 (2020) and repurposing of FDA-approved drugs are studied for the inhibition of SARS-CoV-2 (Riva, et al., Nature (2020), Maisonnasse, et al., Nature (2020), Gordon, et al., Nature 583, 459-468 (2020)).

Since the emergence of COVED-19, many clinical trials have been carried out for repurposing the approved drugs including chloroquine, arbidol, camostat, remdesivir, ribavirin, and lopinavir/ritonavir against SARS-CoV-2 (Dong, et al., Drug Discov Ther 14, 58-60 (2020)). Chloroquine probably interfered with endocytic pathway to broadly inhibit SARS-CoV-2 (Wang, et al., Cell Res (2020)), SARS-CoV (Vincent, et al., Virol J 2, 69 (2005)), influenza virus, Ebola and other viruses in vitro (Rebeaud & Zores, Front Med (Lausanne) 7, 184 (2020)). However, its clinical efficacy is limited in COVID-19 patients (Borba, et al., JAMA Netw Open 3, e208857 (2020), Erickson, et al., Toxicol Commun 4, 40-42 (2020), Hashem, et al., Travel Med Infect Dis 35, 101735 (2020) due to its potential cardiac side effects and lack of antiviral activity in vivo (Maisonnasse, et al., Nature (2020), Falzarano, et al., Emerg Infect Dis 21, 1065-1067 (2015)). Arbidol, the clinically available drug in China and Russia, is in Phase III trial against influenza in US. Arbidol demonstrated broad-spectrum in vitro antiviral activity against many viruses including influenza virus, coronaviruses, and Ebola (Hulseberg, et al., Journal of virology 93 (2019), Kadam, & Wilson, Proc Natl Acad Sci USA 114, 206-214 (2017)), with an IC50 of 2-20 μg ml−1 against SARS-CoV-2 (Wang, et al., Cell Res (2020), Wang, et al., Cell Discov 6, 28 (2020)). However, the peak serum concentration of arbidol is lower than 2 μg ml⁻¹ within 5 h after administration of usual drug dosage (Deng, et al., Antimicrob Agents Chemother 57, 1743-1755 (2013), Sun, et al., Int J Clin Pharmacol Ther 51, 423-432 (2013)), which might explain the uncertain clinical efficacy of arbidol in SARS-CoV-2 patients (Zhu, et al., J Infect 81, e21-e23 (2020), Lian, et al., Clin Microbiol Infect 26, 917-921 (2020), Li, et al., Med (N Y) (2020)). Camostat mesylate (Camostat), the inhibitor of TMPRSS2 which facilitates virus entry on cell surface, has been showed to inhibit SARS-CoV, SARS-CoV-2 and other viruses (Hoffmann, et al., Cell 181, 271-280 e278 (2020), Zhou, et al., Antiviral Res 116, 76-84 (2015)).

An effective broad-spectrum antiviral will improve patients' outcome and may reduce transmission in the community and hospitals even before the identification of the novel emerging virus and the specific antiviral drug. The ‘one bug-one drug’ approach to antiviral drug is successful for HIV, hepatitis C virus and influenza virus (Vigant et al., Nat Rev Microbiol 13, 426-437 (2015)). However, there is an urgent need for broad-spectrum antivirals for combating emerging and re-emerging new virus outbreaks, such as the SARS-CoV-2, before the new virus is identified or specific antiviral drug is available.

It is an object of the present invention to provide broad spectrum antiviral agents.

It is also an object of the present invention to provide compositions of broad spectrum antiviral agents.

It is still an object of the present invention to provide methods for treating viral infections in a subject in need thereof.

### SUMMARY OF THE INVENTION

Antiviral agents, compositions containing the antiviral agents and methods of use thereof, are provided. The antiviral agents include P9R (SEQ ID NO:2), or P9R-like peptides derived from P9R, characterized in that they “inhibit endosomal acidification” and “peptide-virus binding” as determined by in vitro endosomal acidification and peptide-virus binding assays. In some preferred forms, the antiviral agent is P9R. The antiviral compositions include a therapeutically effective amount of the antiviral agents

The antiviral compositions can be administered to a subject in need thereof, to treat the symptoms associated with a viral infection. Preferably the subject is infected with a respiratory virus, more preferably, a pH-dependent virus that requires endosomal acidification for virus-host membrane fusion. Examples include, but are not limited to the enveloped coronaviruses (SARS-CoV-2, SARS-CoV and MERS-CoV), the pandemic A(H1N1)pdm09 virus, avian influenza A(H7N9) virus, and the non-enveloped rhinovirus.

Disclosed are antiviral agents and antiviral compositions. In some forms, the antiviral agents and compositions inhibit antiviral replication in cells. In some forms, the antiviral agents and compositions inhibit viral entry into cells. In some forms, the antiviral agents and compositions inhibit viral entry into cells and antiviral replication in cells.

In some forms, the antiviral agents comprise a multivalent peptide, where the multivalent peptide comprises three or more copies of one or a combination of peptides selected from the group consisting of P9R (SEQ ID NO:2) and P9R-like peptides derived from P9R, where at least three of the peptides that comprise the multivalent peptide branch from one or more of the peptides that comprise the multivalent peptide.

In some forms, the multivalent peptides comprise six or more copies of the peptides, where at least six of the peptides that comprise the multivalent peptide branch from one or more of the peptides that comprise the multivalent peptide. In some forms, the multivalent peptides comprise eight or more copies of the peptides, where at least eight of the peptides that comprise the multivalent peptide branch from one or more of the peptides that comprise the multivalent peptide.

In some forms, at least three of the peptides that branch from one or more of the peptides that comprise the multivalent peptide branch from a central point in the multivalent peptide. In some forms, at least six of the peptides that branch from one or more of the peptides that comprise the multivalent peptide branch from a central point in the multivalent peptide. In some forms, at least eight of the peptides that branch from one or more of the peptides that comprise the multivalent peptide branch from a central point in the multivalent peptide. In some forms, the peptides that branch from one or more of the peptides that comprise the multivalent peptide branch from a central point in the multivalent peptide.

In some forms, the P9R-like peptides are characterized in that the P9R-like peptide inhibits endosomal acidification and retains virus binding as determined by an in vitro endosomal acidification, optionally compared to a control, and a peptide-virus binding assays. In some forms, the peptides that comprise the multivalent peptide each consist of P9R (SEQ ID NO:2).

In some forms, one or more of the peptides that comprise the multivalent peptide has a net positive charge of at least 5. In some forms, the peptides that comprise the multivalent peptide each has a net positive charge of at least 5. In some forms, one or more of the peptides that comprise the multivalent peptide has a net positive charge of about 5.6. In some forms, the peptides that comprise the multivalent peptide each has a net positive charge of about 5.6. In some forms, one or more of the peptides that comprise the multivalent peptide has a net positive charge of 5.6. In some forms, the peptides that comprise the multivalent peptide each has a net positive charge of 5.6.

In some forms the antiviral agents comprise P9R (SEQ ID NO:2), or a P9R-like peptides derived from P9R. In some forms, the P9R-like peptide is characterized in that the P9R-like peptide inhibits endosomal acidification and retains virus binding as determined by an in vitro endosomal acidification, optionally compared to a control, and a peptide-virus binding assays. In some forms, the antiviral agent consists of P9R (SEQ ID NO:2).

In some forms, the antiviral agent has a net positive charge of at least 5. In some forms, the antiviral agent has a net positive charge of about 5.6. In some forms, the antiviral agent has a net positive charge of 5.6.

In some forms, the antiviral compositions comprise any one or more of the disclosed antiviral agents and a pharmaceutically acceptable carrier. In some forms the antiviral compositions comprise a therapeutically effective amount of any one or more of the disclosed antiviral agents and a pharmaceutically acceptable carrier.

In some forms, the antiviral compositions comprise arbidol, chloroquine, and camostat.

In some forms, the composition inhibits antiviral replication in the subject. In some forms, the composition is a unit dosage form. In some forms, the unit dosage form is selected from the group consisting of a table or capsule. In some forms, the composition is in a form suitable for intranasal or pulmonary delivery. In some forms, the unit dosage form is an injectable, where the composition further comprises a pharmaceutically acceptable carrier for injection to a human.

Also disclosed are methods of treating a viral infection in a subject in need thereof. In some forms, the method comprises administering to the subject an effective amount of any of the disclosed the antiviral agent s or any of the disclosed antiviral compositions.

In some forms, the infection is caused by a respiratory virus. In some forms, the infection is caused by a pH-dependent virus that requires endosomal acidification for virus-host membrane fusion. In some forms, the infection is caused by zika virus, enterovirus-A7, ebola virus, influenza virus, SARS-CoV-2, SARS-CoV, MERS-CoV, the A(H1N1)pdm09 virus, avian influenza A(H7N9) virus, and the non-enveloped rhinovirus.

In some forms, the composition is administered parenterally or orally. In some forms, the composition is administered intranasally, or by pulmonary administration.

### BRIEF DESCRIPTION OF THE DRAWINGS

FIG. 1A shows the peptide sequences (P9 (SEQ ID NO:1); P9R (SEQ ID NO:2); PA1 (SEQID NO:3); and P9RS (SEQ ID NO:4) and positive charge analyzed by PepCalc of InnovaGen. FIGS. 1B-1H showP9R inhibition viral replication of 2019 new coronavirus (SARS-CoV-2), MERS-CoV, SARS-CoV, H1N1 virus, H7N9 virus, rhinovirus, and parainfluenza 3 virus in cells. Viruses were premixed with different concentrations of P9R or P9 and then infected cells. The antiviral efficiency was evaluated by plaque reduction assay. Infection (%) was calculated by the plaque number of virus treated with peptides being divided by the plaque number of virus treated by BSA. FIG. 1I shows potent antiviral activities of P9R against viruses by measuring the viral RNA copies in supernatants at 24 h post infection when viruses were treated by P9R or BSA (50-100 μg ml⁻¹). FIG. 1J shows the cytotoxicity of P9R in MDCK, Vero E6 and A549 cells. * indicates P<0.05 and ** indicates P<0.01 when IC₅₀ of P9R compared with that of P9. P values were calculated by the two-tailed Student's t test. Data are presented as mean±SD from at least three independent experiments.

FIG. 2A shows the quantification of red fluorescence of endosomal acidification in MDCK cells treated by peptides. The red fluorescence intensity was calculated from 10 random microscope fields. FIG. 2B shoes the antiviral activities of 25 μg ml⁻¹ of P9R, P9RS, and PA1 against SARS-CoV-2 and A(H1N1)pdm09 virus were measured by plaque reduction assay. Plaque number (%) of peptide-treated virus was normalized to BSA-treated virus. FIG. 2C shows P9R and PA1 binding to SARS-CoV-2 and A(H1N1)pdm09 virus. Viruses binding peptides were detected by ELISA and RT-qPCR. ** indicates P<0.01 when compared with P9R. P values were calculated by the two-tailed Student's t test.

FIG. 3A shows P9R binding to SARS-CoV-2 and A(H1N1)pdm09 could be reduced by PA1. Virus was pretreated by PA1 or BSA, and then the treated virus binding to the indicated peptides were measured by RT-qPCR. ** indicates P<0.01 when compared virus treated by BSA. (c) P9R could inhibit viral RNP release into nuclei. H1N1 virus was pretreated by BSA, P9R or bafilomycin A1 (BA1), and then MDCK cells were infected with the treated virus. Images of viral NP (green) and cell nuclei (blue) were taken at 3.5 h post infection. FIGS. 3B-3D include data showing P9R could broadly bind to MERS-CoV, H7N9 virus, and rhinovirus. The relative RNA copy of virus binding to peptides was normalized to the virus binding to P9R. * indicates P<0.05. ** indicates the P<0.01 when compared with P9R. P values were calculated by the two-tailed Student's t test. FIG. 3E shows Peptides binding to virus and viral proteins. For peptides binding to SARS-CoV (left panel). SARS-CoV was incubated with the indicated peptides on ELISA plate for 1 h. The unbinding virus was washed away and the binding SARS-CoV was quantified by RT-qPCR. Relative RNA copy (%) was normalized to RNA copy of virus binding to P9R. Peptides binding to H1N1 HA1 protein (middle panel). Peptides binding to MERS-CoV S protein (left panel). Peptides were coated on ELISA plates. The H1N1 HA and MERS-CoV S proteins binding to peptides were measured by ELISA assay. * indicates P<0.05. ** indicates P<0.01 when compared with P9R. P values were calculated by the two-tailed Student's t test.

FIG. 4A shows P9R (50 μg/dose) therapeutic efficacy on mice infected by A(H1N1) virus as that of zanamivir (50 μg/dose). PBS, zanamivir, PA1, P9R, or P9 were intranasally inoculated to mice at 6 h post infection and two more doses were administrated to mice in the following one day. Five mice in each group were included. FIG. 4B shows the body weight change of infected mice corresponding to (FIG. 4A). FIG. 4C shows the effect of low doses of P9R on mice infected by A(H1N1)pdm09 virus compared to P9. PBS (n=10), P9-25 (25.0 μg/dose, n=5), P9-12.5 (12.5 μg/dose, n=5), P9R-25 (25.0 μg/dose, n=10), and P9R-12.5 (12.5 μg/dose, n=10) were intranasally inoculated to mice at 6 h post infection and two more doses were administrated to mice in the following one day. FIG. 4D shows the body weight change of infected mice corresponding to (FIG. 4C). P values were calculated by Gehan-Breslow-Wilcoxon test.

FIG. 5A shows the procedure of drug-resistance assay for zanamivir and P9R. A(H1N1) virus was passaged in the presence of indicated concentrations of zanamivir and P9R. ND, not detected because the high resistant H1N1 virus against zanamivir was generated before P16. FIG. 5B shows Zanamivir inhibition of parent A(H1N1) virus (P0). The IC₅₀ of zanamivir against parent H1N1 was 35 nM. FIG. 5C shows the antiviral efficiency of zanamivir against passaged A(H1N1) virus in the presence of zanamivir. FIG. 5D shows the antiviral efficiency of P9R against passaged A(H1N1) virus in the presence of P9R. Passaged viruses were premixed with zanamivir (nM) or P9R (μg ml⁻¹) for infection. Supernatants were collected at 24 h post infection. Viral titers in the supernatants were determined by RT-qPCR. The relative replication (%) was normalized to the corresponding passaged viruses without treatment. Data are presented as mean±SD of three independent experiments.

FIG. 6A is a schematic figure of single P9R binding to single viral particle and branched P9R (8P9R) cross-linking viruses together. FIG. 6B is bar graph showing the binding of 8P9R and P9R to SARS-CoV-2 and H1N1 viruses. Peptides coated on ELISA plates could capture virus particles which were then quantified by RT-qPCR. P9RS was the negative control peptide with no viral binding ability. Data are presented as mean±SD of three independent experiments. FIG. 6C is a bar graph showing relative binding of control and different peptides to SARS-CoV-2 and H1N1. SARS-CoV-2 was pretreated with the indicated peptides for plaque reduction assay. Data are presented as mean±SD of four independent experiments. FIG. 6D is a bar graph showing SARS-CoV-2 plaque number (%) for P9R and 8P9R peptides. SARS-CoV-2 was treated by indicated peptide (25 μg ml⁻¹) during viral inoculation. Viral RNA copies were detected by RT-qPCR at 24 host post infection in the supernatant of Vero-E6 cells. Data are presented as mean±SD of three independent experiments. FIG. 6E is a line graph showing PFU/ml as a function of time. SARS-CoV-2 was treated by peptides (50 μg ml⁻¹) at 6 h post infection. Viral titers were measured at the indicated time by plaque assay. Data are presented as mean±SD of three independent experiments. FIG. 6F is a bar graph showing the results of a hemolysis assay of 8P9R in turkey red blood cells (TRBC). TRBC were treated by the indicated concentration of 8P9R. Hemolysis (%) was normalized to TRBC treated by Triton X-100. Data are presented as mean±SD three independent experiments. P values are calculated by two-tailed student t test. * indicates P<0.05. ** indicates P<0.01. FIG. 6G is a bar graph showing antiviral activity of P9R in PB buffer. SARS-CoV-2 was pretreated by the indicated concentrations of P9R in 30 mM phosphate buffer (PB). After 45 min incubation, plaque reduction assay was used to measure the antiviral activity of P9R. Plaque number of virus treated by P9R was normalized to the plaque number of untreated virus. Data are presented as mean±SD of five independent experiments. FIG. 6H is a bar graph showing cytotoxicity of 8P9R in Vero-E6. Vero-E6 cells were cultured in the presence of indicated concentrations of 8P9R in DMEM with 1% FBS medium. After 24 h culture, MTT assay was used to measure the cell viability. Data are presented as mean±SD from three independent experiments. FIG. 6I is a bar graph showing antiviral activity of 8P9R against H1N1, parainfluenza virus 3 and human rhinovirus. H1N1, parainfluenza virus 3 and human rhinovirus were premixed with 8P9R (25 μg/ml) or PBS (Mock) at room temperature for 45 min. Then MDCK cells were infected with the treated influenza virus. LLC-MK2 cells were infected with the treated parainfluenza virus. RD cells were infected with the treated rhinovirus. The infection (%) for H1N1 was the plaque number of 8P9R-treated virus normalized to the plaque number of mock-treated virus, and the infection (%) for parainfluenza virus 3 and rhinovirus was the viral RNA copies of 8P9R-treated virus normalized to the RNA copies of PBS-treated virus. Data were presented as mean±SD of three independent biological samples.

FIG. 7A is a line graph showing the effect of 8P9R on antiviral activity of arbidol against SARS-CoV-2 in Vero-E6 cells (n=5). Virus infected cells at the presence of the indicated concentrations of arbidol (Ar) or Ar+8P9R (3.1 μg ml⁻¹) or Ar+8P9R (1.6 μg ml⁻¹). FIG. 7B is a bar graph showing the effect of 8P9R on antiviral activity of arbidol compared to arbidol alone (n=4). SARS-CoV-2 was treated by the indicated Ar-0.2 (0.2 μg ml⁻¹), 8P9R-3.1 (3.1 μg ml⁻¹), Ar+8P9R, or PBS (Mock). FIG. 7C is a bar graph showing the effect of mock, 8P9R, and arbidol on SARS-CoV-2 plaque formation (PFU/ml). SARS-CoV-2 (10⁶ PFU ml⁻¹) were treated by 25 μg ml⁻¹ arbidol, or 8P9R (n=3). Then virus was serially diluted to detect the viral titer by plaque assay. FIG. 7D is a bar graph showing the effect of arbidol, BA1, and mock on SARS-CoV-2 on relative viral RNA copy (%) over time post-infection. SARS-CoV-2 was treated at the indicated time of post infection by the indicated drugs (n=3). Viral titers (a, b and d) were measured by RT-qPCR at 24 h post infection. Data are presented as mean±SD from 3-5 independent experiments. P values are calculated by two-tailed student t test. FIG. 7E is a bar graph showing effect of 8P9R on antiviral activity of arbidol. SARS-CoV-2 was cultured in the presence of indicated arbidol (Ar-0.2, 0.2 μg ml⁻¹), 8P9R-1.6 (1.6 μg ml⁻¹) or the combination of Ar+8P9R. Viral titers in supernatants were measured at 24 h post infection by RT-qPCR. Data are presented as mean±SD from four independent experiments. P value was calculated by two-tailed student t test. FIG. 7F is a bar graph showing the effect of arbidol on the antiviral activity of 8P9R. SARS-CoV-2 was cultured in the presence of indicated arbidol (Ar-12.5, 12.5 μg ml⁻¹), 8P9R-0.8 (0.8 μg ml⁻¹) or the combination of Ar+8P9R. Viral titers in supernatants were measured at 24 h post infection by RT-qPCR. Data are presented as mean ±SD from three independent experiments. P value was calculated by two-tailed student t test. FIG. 7G is a bar graph showing the effect of arbidol on viral attachment of SARS-CoV-2 in Vero-E6 cells. SARS-CoV-2 was pretreated by arbidol (Ar, 25 μg ml⁻¹) or 0.1% DMSO (Mock) and then was added to Vero-E6 cells at 4° C. for attachment. One hour later, the unattached virus was washed away. The attached virus was measured by RT-qPCR. Data are presented as mean±SD from three independent experiments.

FIG. 8A is a bar graph showing the effect of chloroquine (Chl) on the activity of arbidol against SARS-CoV-2 compared to arbidol alone (0.2 μg ml⁻¹, Ar-0.2) (n=4). SARS-CoV-2 was treated by the indicated Ar-0.2, Chl-3.1 (3.1 μg ml⁻¹), or Ar+Chl. FIG. 8B is a bar graph showing the effect of chloroquine (Chl) on the activity of arbidol against SARS-CoV-2 compared to arbidol alone (0.4 μg/ml, Ar-0.4) (n=3). SARS-CoV was treated by the indicated Ar-0.4, Chl-6.3 (6.3 μg ml⁻¹), or Ar+Chl. FIG. 8C is a bar graph showing the antiviral activity of indicated drugs or drug combinations against SARS-CoV in mice. Mice were inoculated with SARS-CoV (5×10 3 PFU). 8P9R (intranasal 0.5 mg kg⁻¹, n=8), arbidol (Ar, oral 30 mg kg⁻¹, n=8), chloroquine (Chl, oral 40 mg kg⁻¹, n=6), camostat (Cam, intranasal 0.3 mg kg⁻¹, n=5), Ar+Chl (n=6), Ar+Cam (n=6), Chl+Cam (n=6), Ar+Chl+Cam (n=5) and mock (n=12) were given to mice at 8 h post infection. Viral loads were measured by plaque assay at 48 h post infection. FIGS. 8D-8E are bar graphs showing the antiviral activity of 8P9R (12.5 μg ml⁻¹), arbidol (12.5 μg ml⁻¹), and chloroquine (12.5 μg ml⁻¹) in Vero-E6 (8D, n=4) and Calu-3 (8E, n=5) cells. Viral RNA copies in cell supernatants were measured by RT-qPCR at 24 h post infection. FIG. 8F is a bar graph showing the antiviral activity of indicated drugs or drug combinations against SARS-CoV-2 in hamsters. Hamsters were inoculated with SARS-CoV-2 (5×10³ PFU). Mock (n=9), 8P9R (n=4), Ar+Chl+Cam (n=6), Chl+Cam (n=6), Ar+Cam (3), Cam (n=5), Ar (n=3), and Chl (n=4) were given to hamsters at 8 h post infection. Viral loads were measured by plaque assay at 48 h post infection. Data are presented as mean±SD. P values are calculated by two-tailed student t test. FIG. 8G is a bar graph showing the effect of camostat on SARS-CoV replication in mice. Mice were intranasally inoculated with SARS-CoV (2×10³ PFU). Camostat (Cam: 15 mg kg⁻¹, n=3) or Mock (n=3) was orally inoculated to mice at 8 h post infection. Two more doses were given to mice in the following one day. Lung tissues were collected at 2-day post infection. Viral loads in lungs were measure by plaque assay. Data are presented as mean±SD.

### DETAILED DESCRIPTION OF THE INVENTION

### I. Definitions

“Aerosol” as used herein refers to any preparation of a fine mist of particles, which can be in solution or a suspension, whether or not it is produced using a propellant.

An “emulsion” is a composition containing a mixture of non-miscible components homogenously blended together.

“Hydrophilic” as used herein refers to substances that have strongly polar groups that readily interact with water.

“Hydrophobic” as used herein refers to substances that lack an affinity for water; tending to repel and not absorb water as well as not dissolve in or mix with water.

“Lipophilic” as used herein refers to compounds having an affinity for lipids.

“Parenteral administration”, as used herein, means administration by any method other than through the digestive tract or non-invasive topical or regional routes.

“Patient” or “subject” to be treated as used herein refers to either a human or non-human animal.

“Pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Pharmaceutically acceptable salt”, as used herein, refers to derivatives of the compounds defined herein, wherein the parent compound is modified by making acid or base salts thereof.

“Therapeutically effective” or “effective amount” as used herein means that the amount of the composition used is of sufficient quantity to ameliorate one or more causes or symptoms of a disease or disorder. Such amelioration only requires a reduction or alteration, not necessarily elimination. As used herein, the terms “therapeutically effective amount” “therapeutic amount” and “pharmaceutically effective amount” are synonymous. One of skill in the art can readily determine the proper therapeutic amount.

A “subject” or “patient” refers to a human, primate, non-human primate, laboratory animal, farm animal, livestock, or a domestic pet.

### II. Compositions

Compositions having dual-antiviral mechanisms of cross-linking viruses to stop viral entry (mediated by TMPRSS2 for SARS-CoV-2) and of reducing endosomal acidification to inhibit viral entry through endocytic pathway are provided. In some preferred forms, the disclosed compositions include a potent antiviral peptide P9R (NGAICWGPCPTAFRQIGNCGRFRVRCCRIR; SEQ ID NO:2), derived from mouse β-defensin-4 and P9 (NGAICWGPCPTAFRQIGNCGKFKVRCCKIR; (SEQ ID NO:1). Mechanistic studies showed that positively charged P9R broadly inhibits viral replication by binding to different viruses and then inhibiting virus-host endosomal acidification to prevent the endosomal release of pH-dependent viruses. P9R (not only binding to viruses but also inhibiting endosomal acidification), PA1 (only binding to viruses) and P9RS (only inhibiting endosomal acidification) were used to identify and confirm the novel antiviral mechanism of alkaline peptides. The antiviral activity of alkaline peptide could be enhanced by increasing the positive charge of peptide and required both of binding to viruses and inhibiting endosomal acidification. The peptide can be monovalent or a multivalent having, for example, 2, 3, 4, 5, 6, 7, 8, 9, 10, 11, 12, or more copies of the antiviral peptide.

Additionally, or alternatively, the compositions can include two or more active agents selected from one or more small molecule drugs optionally in combination with P9R, or a derivative thereof, either as a monopeptide or a multivalent peptide, that together can inhibit two entry pathways of a virus such as SAR-CoV-2.

A. Antiviral Active Agents

Antiviral peptide and small molecule active agents, and combinations thereof are provided.

1. Antiviral Peptides

The disclosed antiviral peptide preferably consists of the sequence of P9R. However, the antiviral peptide can include peptides derived from P9R, so long as the amino acids at positions 21, 23 and 28 are positively charge amino acids. Thus, the peptide can have the same amino acid sequence as P9R, with the arginine at positions 21, 23 and 28 replaced with a positively charged amino acid such as lysine or histidine. It is essential however that any modification of the P9R structure ensures that resulting peptide retains inhibition of endosomal acidification and retains virus binding to the same extent as P9R. Therefore, useful P9R-derived peptides (herein, P9R-like peptides) possess the properties of “inhibition of endosomal acidification” and “virus binding”. It is within the abilities of one of ordinary skill in the art to vary the amino acids in P9R and test for the required activities (inhibition of endosomal acidification” and “virus binding”) as shown in the examples of this application.

“A virus-binding assay” includes the following steps: Dissolving the Peptides (0.1 μg per well) in H₂O and coating onto ELISA plates, then incubating at 4° C. overnight. Then, 2% BSA is added to block plates at 4° C. overnight. For virus binding to peptides, viruses are diluted in phosphate buffer and then added to ELISA plate for binding to the coated peptides at room temperature for 1 h. After washing the unbinding viruses, the binding viruses are lysed by RLT buffer of RNeasy Mini Kit (Qiagen, Cat #74106) for viral RNA extraction. Viral RNA copies of binding viruses were measured by RT-qPCR.

“An Endosomal acidification assay” can include detecting endosomal acidification with a pH-sensitive dye (pHrodo Red dextran, Invitrogen, Cat #P10361) according to the manufacturer's instructions as previously described but with slight modification (Zhao et al., Nat Commun 9, 2358 (2018)). First, MDCK cells are treated with BSA (25.0 μg ml⁻¹), P9 (25.0 μg ml⁻¹), P9R (25.0 μg ml⁻¹), PA1 (25.0 μg ml⁻¹), or P9RS (25.0 μg ml⁻¹) at 4° C. for 15 min. Second, MDCK cells are added with 100 vg ml⁻¹ of pH-sensitive dye and DAPI and then incubated at 4° C. for 15 min. Before taking images, cells are further incubated at 37° C. for 15 min and then cells were washed twice with PBS. Finally, PBS is added to cells and images were taken immediately with confocal microscope (for example, Carl Zeiss LSM 700, Germany).

Therefore, the P9R-derived peptide should have an overall net positive charge of at least 5, preferably at least 5.6, and preferably, does not include amino acid modifications as shown for P9RS (SEQ ID NO:4). Preferably, also, the P9R-derived peptide does not include an introduction of additional amino acid residues at the C-terminal arginine.

Amino acid substitutions in P9R to obtain P9R-like peptides preferably include conservative amino acid substitutions.

Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

It is understood, however, that substitutions at the recited amino acid positions can be made using any amino acid or amino acid analog. For example, the substitutions at the recited positions can be made with any of the naturally occurring amino acids (e.g., alanine, aspartic acid, asparagine, arginine, cysteine, glycine, glutamic acid, glutamine, histidine, leucine, valine, isoleucine, lysine, methionine, proline, threonine, serine, phenylalanine, tryptophan, or tyrosine).

P9R-derived peptides can have, for example, 70%, 75%, 80%, 85%, 90%, 95%, 96%, 97%, 98%, 99%, or more sequence identity to SEQ ID NO:2.

The antiviral peptide can be in a monovalent form, multivalent form, or a combination thereof. A multivalent peptide can include, for example, 2, 3, 4, 5, 6, 7, 8, 9, 10, 11, 12, or more copies of the antiviral peptide. Multivalent forms can be, for example, branched peptides. Branched peptides typically include one or more isopeptide bonds. An isopeptide bond is an amide bond that can form for example between the carboxyl group of one amino acid and the amino group of another. At least one of these joining groups is part of the side chain of one of these amino acids. Common branching strategies include branching at Asp/Glu, Lys, and Ser/Thr side chains. For example, a lysine backbone can be used as a scaffolding core to support the formation of as many as 8 or 12 branches with varying or the same peptide sequences. See, e.g., U.S. Published Application No. 20180333481 and Jones, et al., Molecular Psychiatry, 25:2994-3009 (2020). Additionally or alternatively, a function group of side-chain benzyl ester can be employed as the precursor of hydrazide, which can be used to assemble a branch peptide by native chemical ligation or direct amidation (Liu, et al., Meth. In Molec. Biology, 2103:189-198 (2019) DOI: 10.1007/978-1-0716-0227-0_12). Genetically encoded tags can also be used to make proteins branch in a variety of shapes (Zhang, et al., J. Am. Chem. Soc., 135, 37, 13988-13997 (2013), DOI: 10.1021/ja4076452). See also Brunetti, et al., Pept Sci., 110:e24089 (2018) https://doi.org/10.1002/pep2.24089. By placing branch-forming residues at different positions in a protein chain, practitioners can control branch points and shape of a multivalent peptide.

A branched multivalent peptide can consist of copies of an antiviral peptide sequence, particularly where the peptide sequence already includes suitable branch points. Alternatively, some or all of the peptides in the multivalent form can include one or more amino acid insertions or modifications to facilitate branching.

Thus, in some forms, the antiviral agent includes a multivalent peptide, wherein the multivalent peptide includes e.g., three, four, five, six, seven, eight, or more copies of one or a combination of peptides selected from the group consisting of P9R (SEQ ID NO:2) and P9R-like peptides derived from P9R.

In some forms, at least four, five, six, seven, eight, or more of the peptides that form the multivalent peptide branch from one or more of the peptides that include the multivalent peptide.

In some forms, at least four, five, six, seven, eight, or more of the peptides that branch from one or more of the peptides that form the multivalent peptide branch from a central point in the multivalent peptide.

The multivalent P9R-like peptides can be characterized in that they inhibit endosomal acidification and retain virus binding as determined by an in vitro endosomal acidification, optionally compared to a control, and a peptide-virus binding assays.

In some forms, one or more, or in some cases all, of the peptides that form the multivalent peptide has/have a net positive charge of at least 5 such as about 5.6 or 5.6. In some particular forms, one or more, or in some cases all, of the peptides that form the multivalent peptide each include or consist of P9R (SEQ ID NO:2).

In some forms one or more of the peptides that form that multivalent peptide include the addition or modification of one or more residues or moieties to facilitate branching, including, but not limited to, amino acid residue(s) with Asp/Glu, Lys, and Ser/Thr side chains, or other residues or modifications capable of forming isopeptide bonds. In some particular forms, such residues are added or inserted into one or more copies SEQ ID NO:2, or a derivative thereof. The additions, substitution, or other modification can be at one or more of the N-terminus, C-terminus, or interior of the peptide.

In some forms, the multivalent peptide include or consists of three, four, five, six, seven, eight, or more copies of one or a combination of peptides selected from the group consisting of P9R (SEQ ID NO:2) and P9R-like peptides derived from P9R, branched or otherwise linked by a lysine scaffold.

Preferably, the multivalent peptide is effective to increase cross-linking of viruses and can enhance blockage of viral on cell surface through a TMPRSS2-mediated pathway, more preferably while simultaneously reducing endosomal acidification to block viral entry through endocytic pathway. In some forms, the multivalent peptide is potent at one or more of these mechanisms that its monovalent counterpart.

2. Antiviral Combinations

In some forms, the disclosed compositions and methods include two or more active agents. Typically, the antiviral agents are used in a combination that simultaneously block two entry pathways of a virus, e.g., a coronavirus such as SARS-CoV-2. ACE2 and TMPRSS2 are individually expressed in some human cell types or co-expressed in other cell types (Sungnak, et al., Nat Med 26, 681-687 (2020)), thus, in some preferred forms, the two entry pathways are ACE2-mediated and TMPRSS2-mediated pathways.

The results presented in the experiments in Example 2 below show that the approach of simultaneous inhibition of virus entry through the endocytic pathway and the surface fusion pathway mediated by TMPRSS2 can improve antiviral effect. More particularly, results show that endosomal acidification inhibitors (e.g., 8P9R or chloroquine) could significantly enhance the antiviral efficiency of arbidol, which was found to inhibit virus-cell membrane fusion, at a clinically achievable concentration against SARS-CoV-2 and SARS-CoV replication in Vero-E6 cells, where coronaviruses mainly enter cells through endocytic pathway. A more than additive mechanism study indicated that 8P9R or chloroquine could elevate endosomal pH which enhances the efficiency of arbidol in blocking virus-host cell fusion mediated by spike and ACE2. To block the two entry pathways of coronavirus, arbidol and chloroquine were combined with comastat which inhibits TMPRSS2 to prevent SARS-CoV-2 fusion on cell surface. Results showed significant antiviral activity against SARS-CoV-2 in hamsters and SARS-CoV in mice. This drug combination had a similar inhibitory effect as the dual-functional 8P9R in the treatment of SARS-CoV-2 and SARS-CoV animal models. In contrast, the single use of arbidol or chloroquine did not show antiviral efficacy in mice and hamsters. Given that all these three drugs are broad-spectrum antivirals, this combination can play important roles in controlling respiratory virus infection with similar entry pathways. The identification of the dual-functional 8P9R and the triple combination of clinical drugs shows that targeting both entry pathways of coronavirus is a viable approach to reduce SARS-CoV-2 replication in vivo.

Thus, in some forms, the compositions and methods include at least two active agents, wherein the agents in combination accomplished two, most preferably all three of: a reduction in endosomal acidification and/or elevate endosomal pH, a reduction in virus-host cell fusion mediated by spike and ACE2, and a reduction in virus-host cell fusion mediated by TMPRSS2. In some forms, the two or more active agents include arbidol in combination with monovalent or multivalent P9R or a derivative thereof, or chloroquine, optionally in further combination with comastat. Particularly preferred combinations are arbidol in combination with monovalent or multivalent P9R or derivative thereof optionally in further combination with comastat, and arbidol in combination with chloroquine and comastat.

In some forms, the combination of two or more active agent agents achieves a result greater than when the individual agents are administered alone or in isolation. For example, in some forms, the result achieved by the combination is partially or completely additive of the results achieved by the individual components alone. In the most some preferred forms, the result achieved by the combination is more than additive of the results achieved by the individual components alone.

In some forms, the effective amount of one or both agents used in combination is lower than the effective amount of each agent when administered separately. In some forms, the amount of one or both agents when used in the combination therapy is sub-therapeutic when used alone.

Arbidol has been formulated as tablets, capsules and granules, in dosages of 50 mg and 100 mg. For the treatment of influenza, children older than two years and adults have used, e.g., mg to 200 mg of arbidol orally, four times a day (every six hours) for five days (Huang, et al., Cochrane Database Syst Rev., 2017(2): CD011489 (2017)). For prophylaxis during direct contact with people with influenza, children older than two years and adults use 50 mg to 200 mg arbidol orally, once a day for 10 to 14 days. In a study for the treatment of COVID-19, arbidol was administered at a preventative dosage of 200 mg qd po, or a therapeutic dosage of 600 mg qd po (Yang, et al., Frontiers in Public Health, 8:249 (2020).

Chloroquine has been administered to treat COVID-19 at wide range of dosages and treatment regimens some of which include up to as much as 1,500 mg and 1,200 mg in a single day (Karalis, et al., Saf Sci. 129: 104842 (2020)). An exemplary regimen is 500 mg twice a day for 10-14 days.

A dosage regimen of 600 mg (200 mg, three times) of camostat mesilate daily has been proposed as a therapy from treatment SARS-CoV-2 infection (Uno, Intern Emerg Med., pg. 1-2 doi:10.1007/s11739-020-02345-9 (2020), Bittmann, et al., Biomed J Sci & Tech Res 27(3) (2020). BJSTR. MS.ID.004519).

Ardidol, chloroquine, and/or camostat can be administered at known clinical dosages, or may also be effective at reduced dosages. Thus, in some forms, the ardidol, chloroquine, and/or camostat are administered at known dosages and/or regimens such as those discussed herein, and in references cited herein or otherwise known in the art. In some forms, the dosage of one or more the drugs, when used in the disclosed combinations, is lower than the dosages and/or regimens discussed herein, and in references cited herein or otherwise known in the art.

The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

Thus, a treatment regimen of a combination therapy can include one or multiple administrations of each active agent. In certain forms, the two or more agents are administered simultaneously in the same or different pharmaceutical compositions.

In some forms, two or more active agents are administered sequentially, typically, in two or more different pharmaceutical compositions. The different active agents be administered hours or days apart. The additive or more than additive result may be evident after one day, two days, three days, four days, five days, six days, one week, or more than one week following administration.

Dosage regimens or cycles of the agents can be completely or partially overlapping, or can be sequential. In some forms, all such administration(s) of one agent occurs before or after administration of the second and/or third agent. Alternatively, administration of one or more doses of the one or more agents can be temporally staggered.

An effective amount of each of the agents can be administered as a single unit dosage (e.g., as dosage unit), or sub-therapeutic doses that are administered over a finite time interval. Such unit doses can be administered on a daily basis for a finite time period, such as up to 3 days, or up to 5 days, or up to 7 days, or up to 10 days, or up to 15 days or up to 20 days or up to 25 days, are all specifically contemplated.

B. Formulations

The peptides and other active agents disclosed herein described herein can be formulated for enteral, parenteral, or pulmonary administration. In some preferred forms, the peptide and optionally other active agents, is formulated for pulmonary administration.

The disclosed active agents including e.g., monovalent and/or multivalent P9R (e.g., 8P9R), or peptides derived therefrom, arbidol, chloroquine and/or camostat each alone or in any combination can be combined with one or more pharmaceutically acceptable carriers and/or excipients that are considered safe and effective and can be administered to an individual without causing undesirable biological side effects or unwanted interactions. The carrier is all components present in the pharmaceutical formulation other than the active ingredient or ingredients.

The active agents each alone or in any combination can also be formulated for use as a disinfectant, for example, in a hospital environment.

1. Pulmonary Formulations

In some forms, the one or more of the active agents is formulated for pulmonary delivery, such as intranasal administration or oral inhalation.

The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions including low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the sub epithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm3, porous endothelial basement membrane, and it is easily accessible.

Carriers for pulmonary formulations can be divided into those for dry powder formulations and for administration as solutions. Aerosols for the delivery of therapeutic agents to the respiratory tract are known in the art. Aerosols can be produced using standard techniques, such as ultrasonication or high-pressure treatment. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions can include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

Solvents that are low toxicity organic (i.e. nonaqueous) class 3 residual solvents, such as ethanol, acetone, ethyl acetate, tetrahydofuran, ethyl ether, and propanol can be used for the formulations. The solvent is selected based on its ability to readily aerosolize the formulation. The solvent should not detrimentally react with the P9R (or P9R-like peptides). An appropriate solvent should be used that dissolves the compounds or forms a suspension of P9R (or P9R-like peptides). The solvent should be sufficiently volatile to enable formation of an aerosol of the solution or suspension. Additional solvents or aerosolizing agents, such as freons, can be added as desired to increase the volatility of the solution or suspension.

In some forms, compositions can contain minor amounts of polymers, surfactants, or other excipients well known to those of the art. In this context, “minor amounts” means no excipients are present that might affect or mediate uptake of P9R (or P9R-like peptides) in the lungs and that the excipients that are present are present in amount that do not adversely affect uptake of P9R (or P9R-like peptides) in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PART Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles can be prepared using single and double emulsion solvent evaporation, spray drying, solvent extraction, solvent evaporation, phase separation, simple and complex coacervation, interfacial polymerization, and other methods well known to those of ordinary skill in the art. Particles can be made using methods for making microspheres or microcapsules known in the art. The preferred methods of manufacture are by spray drying and freeze drying, which entails using a solution containing the surfactant, spraying to form droplets of the desired size, and removing the solvent.

The particles can be fabricated with the appropriate material, surface roughness, diameter and tap density for localized delivery to selected regions of the respiratory tract such as the deep lung or upper airways. For example, higher density or larger particles can be used for upper airway delivery. Similarly, a mixture of different sized particles, provided with the same or different EGS can be administered to target different regions of the lung in one administration.

Formulations for pulmonary delivery include unilamellar phospholipid vesicles, liposomes, or lipoprotein particles. Formulations and methods of making such formulations containing nucleic acid are well known to one of ordinary skill in the art. Liposomes are formed from commercially available phospholipids supplied by a variety of vendors including Avanti Polar Lipids, Inc. (Birmingham, Ala.). In some forms, the liposome can include a ligand molecule specific for a receptor on the surface of the target cell to direct the liposome to the target cell.

2. Parenteral Formulations

Active agents can be formulated for parenteral administration. For example, parenteral administration can include administration to a patient intravenously, intradermally, intraarterially, intraperitoneally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants can be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate;

and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation can also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating P9R (or P9R-like peptides) in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

(a) Controlled Release Formulations

The parenteral formulations described herein can be formulated for controlled release including immediate release, delayed release, extended release, pulsatile release, and combinations thereof.

i. Nano- and Microparticles

For parenteral administration, active agents can be incorporated into microparticles, nanoparticles, or combinations thereof that provide controlled release of the active agents. In forms wherein the formulations contains two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropyl cellulose, methylcellulose, and carboxymethyl-cellulose), alginic acid, lactose and talc. Additionally, a pharmaceutically acceptable surfactant (for example, lecithin) can be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

ii. Method of Making Nano- and Microparticles

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some forms, drug in a particulate form is homogeneously dispersed in a water-insoluble or slowly water soluble material. To minimize the size of the drug particles within the composition, the drug powder itself can be milled to generate fine particles prior to formulation. The process of jet milling, known in the pharmaceutical art, can be used for this purpose. In some forms drug in a particulate form is homogeneously dispersed in a wax or wax like substance by heating the wax or wax like substance above its melting point and adding the drug particles while stirring the mixture. In this case a pharmaceutically acceptable surfactant can be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

(b) Injectable/Implantable Formulations

The active agents described herein can be incorporated into injectable/implantable solid or semi-solid implants, such as polymeric implants. In some forms, one or more active agents is incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, active agents can be incorporated into a polymer matrix and molded, compressed, or extruded into a device that is a solid at room temperature. For example, active agents can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the peptides and small molecules from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

3. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art. In forms where the formulation is for oral administration involving transit through the gastrointestinal tract, the formulation is preferably coated to protect the peptide from gastrointestinal enzymes.

Formulations can be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which can include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material can contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

(a) Controlled Release Enteral Formulations

Oral dosage forms, such as capsules, tablets, solutions, and suspensions, can for formulated for controlled release. For example, active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In some forms, one or more active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still some forms, one or more active agents are formulated into a sold oral dosage form, such as a tablet or capsule, and the solid dosage form is coated with one or more controlled release coatings, such as a delayed release coatings or extended release coatings. The coating or coatings can also contain one or more active agents.

i. Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred forms, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred forms, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In some preferred forms, the acrylic polymer is an acrylic resin lacquer such as that which is commercially available from Rohm Pharma under the tradename EUDRAGIT t®. In further preferred forms, the acrylic polymer comprises a mixture of two acrylic resin lacquers commercially available from Rohm Pharma under the tradenames EUDRAGIT® RL30D and EUDRAGIT RS30D, respectively. EUDRAGIT® RL30D and EUDRAGIT RS30D are copolymers of acrylic and methacrylic esters with a low content of quaternary ammonium groups, the molar ratio of ammonium groups to the remaining neutral (meth)acrylic esters being 1:20 in EUDRAGIT RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT RL/RS mixtures are insoluble in water and in digestive fluids. However, multiparticulate systems formed to include the same are swellable and permeable in aqueous solutions and digestive fluids.

The polymers described above such as EUDRAGIT RL/RS can be mixed together in any desired ratio in order to ultimately obtain a sustained-release formulation having a desirable dissolution profile. Desirable sustained-release multiparticulate systems can be obtained, for instance, from 100% EUDRAGIT® RL, 50% EUDRAGIT® RL and 50% EUDRAGIT t® RS, and 10% EUDRAGIT® RL and 90% EUDRAGIT® RS. One skilled in the art will recognize that other acrylic polymers can also be used, such as, for example, EUDRAGIT® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

ii. Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition can be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and can be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials can also be used. Multi-layer coatings using different polymers can also be applied.

The preferred coating weights for particular coating materials can be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition can include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates can also be used. Pigments such as titanium dioxide can also be used. Small quantities of an anti-foaming agent, such as a silicone (e.g., simethicone), can also be added to the coating composition.

### III. Methods of Using

The disclosed methods are based on studies showing that P9R exhibits very broad-spectrum antiviral activities against the enveloped SARS-CoV-2, MERS-CoV, SARS-CoV, A(H1N1)pdm09, A(H7N9) virus, and the non-enveloped rhinovirus. P9R efficiently protects from a viral challenge when administered in vivo, as demonstrated by its protection of mice (following in vivo administration) from lethal A(H1N1)pdm09 virus challenge. P9R did not cause emergency of drug-resistant virus even after A(H1N1)pdm09 virus was passaged in the presence of P9R for 40 passages. Mechanistic studies indicated that the antiviral activity of P9R depended on the direct binding to viruses and the inhibition of virus-host endosomal acidification, which provides a new concept that virus-binding alkaline peptides could broadly inhibit pH-dependent viruses.

Furthermore a dual-functional cross-linking multivalent 8P9R can inhibit two entry pathways (endocytic pathway and TMPRSS2-mediated surface pathway) of SARS-CoV-2 in cells. The endosomal acidification inhibitors (8P9R and chloroquine) can more than additively enhance the activity of arbidol, a spike-ACE2 fusion inhibitor, against SARS-CoV-2 and SARS-CoV in cells. In vivo studies indicate that 8P9R or the combination of repurposed drugs (arbidol, chloroquine and camostat which is a TMPRSS2 inhibitor), simultaneously interfering with the two entry pathways of coronavirus, can significantly suppress SARS-CoV-2 replication in hamsters and SARS-CoV in mice. In the experimental conditions, arbidol, chloroquine or camostat alone, which only targets one entry pathway of coronavirus (Hoffmann, et al., Cell 181, 271-280 e278 (2020), Hoffmann, et al., Nature (2020)), cannot inhibit SARS-CoV-2 and SARS-CoV in vivo. However, the experiments below show that the drug combination (arbidol, chloroquine, and camostat) and a dual-functional 8P9R can block the two entry pathways of coronavirus and are a promising and achievable approach for inhibiting SARS-CoV-2 replication in vivo.

Accordingly, methods are provided for treating a subject infected with a virus, by administering the subjected a formulation containing an effective amount of the disclosed monovalent or multivalent antiviral peptides alone or in combination of additional active agents, e.g., one or more of arbidol, chloroquine or camostat, or the combination of arbidol, chloroquine and camostat in the absence of antiviral peptides, to ameliorate one or more symptoms associated with the viral infection. Exemplary preferred treatments include P9R monovalent peptide or P9R multivalent peptide (e.g., 8P9R) alone or in dual combination with arbidol, and the combination of triple combination of arbidol, chloroquine and camostat, though other combinations are also contemplated as discussed above. In some preferred forms, the treatment is effective to inhibit viral replication in the subject. The subject can be treated with the disclosed peptides and/or other active agents by administering an effective amount of the peptide and/or other active agents to the subject, enterally, by pulmonary or nasal administration, or parenterally (intravenously, intradermally, intraarterially, intraperitoneally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

The virus is preferably a respiratory virus, and more preferably, a pH-dependent respiratory virus. Respiratory viruses are the most frequent causative agents of disease in humans, with significant impact on morbidity and mortality worldwide, mainly in children. Approximately one-fifth of all childhood deaths worldwide are related to acute respiratory infections (ARIs), particularly in impoverished populations of tropical regions, where ARI case-to-fatality ratios can be remarkably higher than in temperate regions of the world. Eight human respiratory viruses circulate commonly in all age groups and are recognized as adapted to efficient person-to-person transmission; the include HRSV (human respiratory syncytial virus), HPIV (human parainfluenza Virus), HRV (human rhinovirus), ADV (adenovirus), HCoV (human coronavirus) (HCoV-NL63, HCoV-HKU1), SARS-CoV, HMP (human metapneumovirus) HPIV (human parainfluenza virus) and HBoV (human bocavirus). HRSV internalization is considered to be pH-independent and may happen either in plasma or in endosomal membranes.

Exemplary viral infections that can be treated with the disclosed formulations include, but are not limited to zika virus, enterovirus-A7, ebola virus, influenza virus, HRSV, HPIV, HRV, ADV, HPIV, HCoV, SARS-CoV-2, MERS-CoV, SARS-CoV, A(H1N1)pdm09, A(H7N9) virus, and the non-enveloped rhinovirus.

The following non-limiting examples further explain the disclosed and claimed compositions and methods.

### EXAMPLES

### Example 1: P9R and P9R-Related Peptides

### Material and Methods

### Cells and Virus Culture

Madin Darby canine kidney (MDCK, CCL-34), Vero E6 (CRL-1586), RD (CCL136), LLC-MK2 (CCL-7), A549 (CCL-185) cells obtained from ATCC (Manassas, VA, USA) were cultured in Dulbecco minimal essential medium (DMEM) or MEM supplemented with 10% fetal bovine serum (FBS), 100 IU ml⁻¹ penicillin and 100 μg ml⁻¹ streptomycin. The virus strains used in this study included 2019 new coronavirus (SARS-CoV-2)(To et al., Clin Infect Dis (2020)), SARS-CoV, MERS-CoV (hCoV-EMC/2012), A/Hong Kong/415742/2009, A/Hong Kong/415742Md/2009 (H1N1) (a highly virulent mouse-adapted strain), A/Anhui/1/2013 (H7N9) (Zhao et al., Sci Rep 6, 22008 (2016)), rhinovirus (To et al., J Clin Virol 77, 85-91 (2016)) and human parainfluenza 3 (ATCC-C243). For in vitro experiments, viruses were cultured in MDCK, Vero E6, RD and LLC-MK2 cells. For animal experiments, H1N1 virus was cultured in eggs as described previously (Zheng et al., Proc Natl Acad Sci U S A 105, 8091-8096 (2008)).

### Design and Synthesis of Peptides

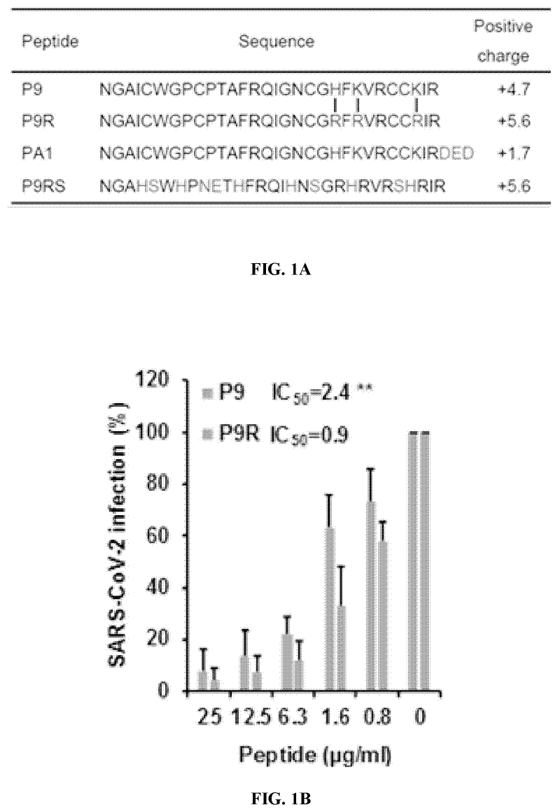

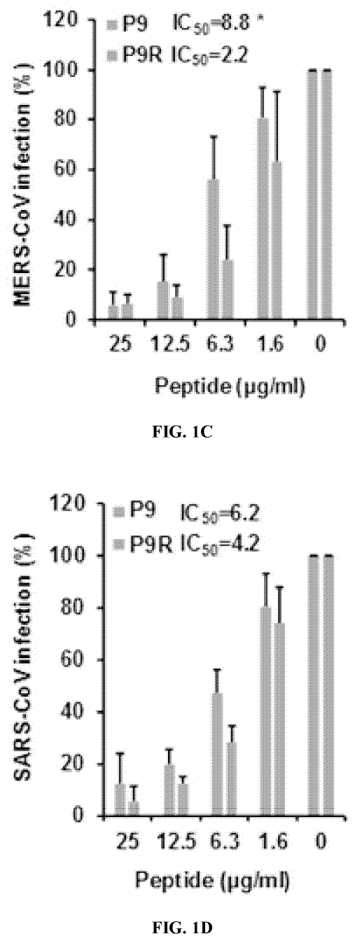

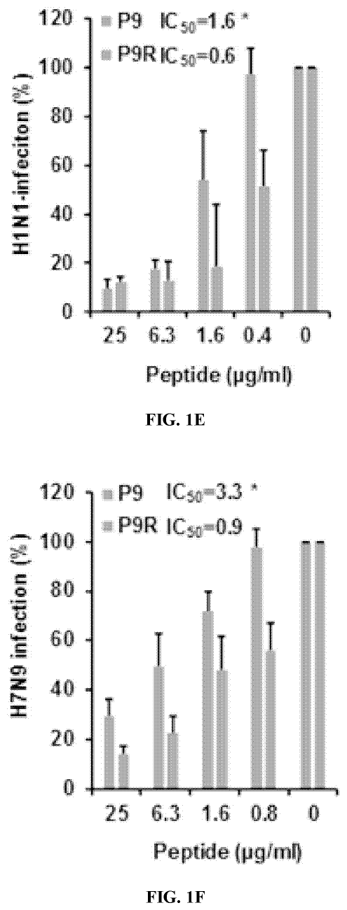

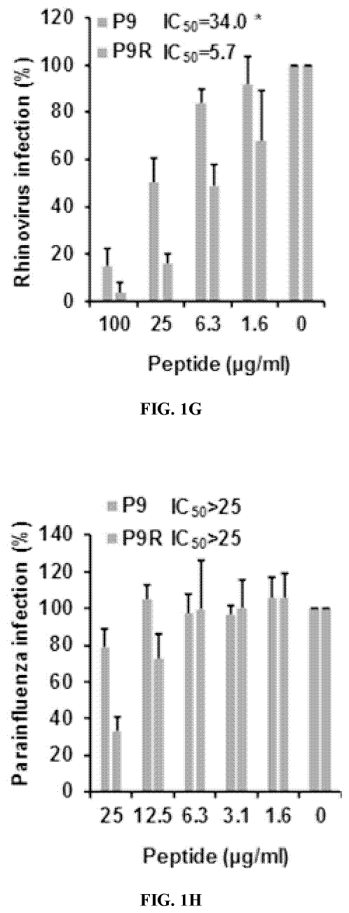

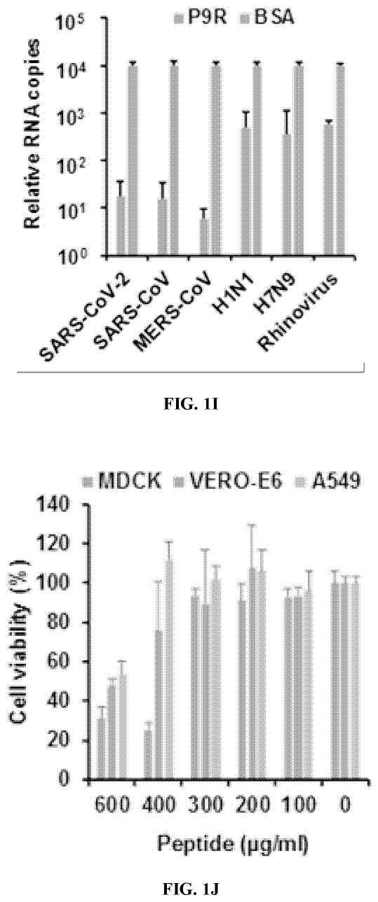

P9, P9R, PA1 and P9RS were designed as shown in FIG. 1A and synthesized by ChinaPeptide (Shanghai, China). The purity of all peptides was>95%. The purity and mass of each peptide were verified by HPLC and mass spectrometry.

### Plaque Reduction Assay

Antiviral activity of peptides was measured using a plaque reduction assay as described previously (Zhao et al., Nat Commun 9, 2358 (2018)). Briefly, peptides were dissolved in 30 mM phosphate buffer containing 24.6 mM Na₂HPO₄ and 5.6 mM KH₂PO₄ at a pH of 7.4. Peptides or bovine serum albumin (BSA, 0.4-50.0 μg ml⁻¹) were premixed with 50 PFU of coronaviruses (SARS-CoV-2, MERS-CoV, and SARS-CoV), influenza viruses (H1N1 virus and H7N9 virus), rhinovirus, or parainfluenza 3 in phosphate buffer at room temperature. After 1 h of incubation, peptide-virus mixture was transferred to Vero E6 for coronaviruses, MDCK for influenza viruses, RD for rhinoviruses, or LLC-MK2 for parainfluenza virus. At 1 h post infection, infectious media were removed and 1% low melting agar was added to cells. Cells were fixed using 4% formalin at 2-4 day post infection. Crystal blue (0.1%) was added for staining, and the number of plaques was counted.

### Antiviral Multicycle Growth Assay

Coronaviruses (SARS-CoV-2, MFRS-CoV, and SARS-CoV), influenza viruses (H1N1 and H7N9 virus) and rhinovirus (0.005 MOI) were premixed with P9R or BSA (50-100 μg ml⁻¹) in phosphate buffer for 1 h. After incubation, coronaviruses were inoculated onto Vero E6. Influenza viruses were inoculated onto MDCK cells. Rhinovirus was inoculated onto RD cells. After 1 h infection, infectious media were removed and fresh media with supplemented P9R or BSA (50-100 μg ml⁻¹) were added to infected cells for virus and cell culture. At 24-30 h post infection, the supernatants of cells were collected for detecting viral RNA copies.

### Cytotoxicity Assay

Cytotoxicity of peptides was determined by the detection of 50% cytotoxic concentration (CC₅₀) using a tetrazolium-based colorimetric MTT assay as described previously (Zhao et al., Sci Rep 6, 22008 (2016)). Briefly, cells were seeded in 96-well cell culture plate at an initial density of 2×10⁴ cells per well in MEM or DMEM supplemented with 10% FBS and incubated for overnight. Cell culture media were removed and then DMEM supplemented with various concentrations of peptides and 1% FBS were added to each well. After 24 h incubation at 37° C., MTT solution (5 mg ml⁻¹, 10 μl per well) was added to each well for incubation at 37° C. for 4 h. Then, 100 μl of 10% SDS in 0.01M HCl was added to each well. After further incubation at room temperature with shaking overnight, the plates were read at OD570 using Victor™ X3 Multilabel Reader (PerkinElmer, USA). Cell culture wells without peptides were used as the experiment control and medium only served as a blank control.

### Peptide-Virus Binding Assay

Peptides (0.1 μg per well) dissolved in H₂O were coated onto ELISA plates and incubated at 4° C. overnight. Then, 2% BSA was used to block plates at 4° C. overnight. For virus binding to peptides, viruses were diluted in phosphate buffer and then were added to ELISA plate for binding to the coated peptides at room temperature for 1 h. After washing the unbinding viruses, the binding viruses were lysed by RLT buffer of RNeasy Mini Kit (Qiagen, Cat #74106) for viral RNA extraction. Viral RNA copies of binding viruses were measured by RT-qPCR.

### ELISA Assay

ELISA assay was done as described previously (Zhao et al., Nat Commun 9, 2358 (2018)). Peptides (0.1 μg per well) dissolved in H₂O were coated onto ELISA plates and incubated at 4° C. overnight. Then, 2% BSA was used to block plates at 4° C. overnight. For HA and S binding, 150 ng HA1 or S in solution I buffer (Sino Biological Inc., Cat #11055-V08H4) was incubated with peptides at 37° C. for 1 h. The binding abilities of peptides to HA1 or S proteins were determined by incubation with rabbit anti-His-HRP (Invitrogen, Cat #R93125, 1: 2,000) at room temperature for 30 min. The reaction was developed by adding 50 μl of TMB single solution (Life Technologies, Cat #002023) for 15 min at 37° C. and stopped with 50 μl of 1 M H2504. Readings were obtained in an ELISA plate reader (Victor 1420 Multilabel Counter; PerkinElmer) at 450 nm.

### Viral RNA Extraction and RT-qPCR

Viral RNA was extracted by Viral RNA Mini Kit (QIAGEN, Cat #52906, USA) according to the manufacturer's instructions. Real-time RT-qPCR was performed as described previously (Zhao et al., Nat Commun 9, 2358 (2018)). Extracted RNA was reverse transcribed to cDNA using PrimeScript II 1^{st} Strand cDNA synthesis Kit (Takara, Cat^{#}6210A) using GeneAmp® PCR system 9700 (Applied Biosystems, USA). The cDNA was then amplified using specific primers (Table 1) for detecting SARS-CoV-2, MERS-CoV, SARS-CoV, H1N1, H7N9, and rhinovirus using LightCycle® 480 SYBR Green I Master (Roach, USA).

TABLE 1
RT-qPCR primers

| | | Oligonucleotide |
|---|---|---|
| Gene | Primer | sequence (5′ to 3′) |
| SARS-COV-2 | S-F | CCTACTAAATTAAATGATCTCTGCTTTACT |
| | | (SEQ ID NO: 5) |
| | S-R | CAAGCTATAACGCAGCCTGTA |
| | | (SEQ ID NO: 6) |
| MERS-COV | NP-F | CAAAACCTTCCCTAAGAAGGAAAAG |
| | | (SEQ ID NO: 7) |
| | NP-R | GCTCCTTTGGAGGTTCAGACAT |
| | | (SEQ ID NO: 8) |
| SARS-COV | NP-F | ACCAGAATGGAGGACGCAAT |
| | | (SEQ ID NO: 9) |
| | NP-R | GCTGTGAACCAAGACGCAGTATTAT |
| | | (SEQ ID NO: 10) |
| H1N1 | M-F | CTTCTAACCGAGGTCGAAACG |
| | | (SEQ ID NO: 11) |
| | M-R | GGC ATTTTGGACAAAKCGTCT A |
| | | (SEQ ID NO: 12) |
| H7N9 | M-F | CTTCTAACCGAGGTCGAAACG |
| | | (SEQ ID NO: 13) |
| | M-R | GGC ATTTTGGACAAAKCGTCT A |
| | | (SEQ ID NO: 14) |
| Rhinovirus | 5′UTR-F | AGCCYGCGTGGCKGCC |
| | | (SEQ ID NO: 15) |
| | 5′UTR-R | AGCCYGCGTGGTGCCC |
| | | (SEQ ID NO: 16) |
| | Probe | HEX-TCCGGCCCCTGAATGYGGCTAA- |
| | | 1ABkFQ (SEQ ID NO: 17) |

For quantitation, 10-fold serial dilutions of standard plasmid equivalent to 10¹ to 10⁶ copies per reaction were prepared to generate the calibration curve. Real-time qPCR experiments were performed using LightCycler® 96 system (Roche, USA).

### Endosomal Acidification Assay

Endosomal acidification was detected with a pH-sensitive dye (pHrodo Red dextran, Invitrogen, Cat #P10361) according to the manufacturer's instructions as previously described but with slight modification (Zhao et al., Nat Commun 9, 2358 (2018)). First, MDCK cells were treated with BSA (25.0 μg ml⁻¹), P9 (25.0 μg ml⁻¹), P9R (25.0 μg ml⁻¹), PA1 (25.0 μg ml⁻¹), or P9RS (25.0 μg ml⁻¹) at 4° C. for 15 min. Second, MDCK cells were added with 100 μg ml⁻¹ of pH-sensitive dye and DAPI and then incubated at 4° C. for 15 min. Before taking images, cells were further incubated at 37° C. for 15 min and then cells were washed twice with PBS. Finally, PBS was added to cells and images were taken immediately with confocal microscope (Carl Zeiss LSM 700, Germany).

### Colocalization Assay of Peptide Binding to Virus in Cells

H1N1 virus was labeled by green Dio dye (Invitrogen, Cat #3898) according to the manufacture introduction. DIO-labeled virus was treated by TAMRA-labeled P9R and TAMRA-labeled P9RS for 1 h at room temperature. Pre-cool MDCK cells were infected by the peptide-treated virus on ice for 15 min and then moved to 37 for incubation for 15 min. Cells were washed twice by PBS and then fixed by 4% formalin for 1 h. Nuclei were stained by DAPI for taking images by confocal microscope (Carl Zeiss LSM 700, Germany).

### Nucleoprotein (NP) Immunofluorescence Assay.

NP staining was carried out as described previously (Zhao et al., Nat Commun 9, 2358 (2018)). MDCK cells were seeded on cell culture slides and were infected with A(H1N1)pdm09 virus at 1 MOI pretreated with BSA (25.0 μg ml⁻¹), bafilomycin A1 (50.0 nM) or P9R (25.0 μg ml⁻¹). After 3.5 h post infection, cells were fixed with 4% formalin for 1 h and then permeabilized with 0.2% Triton X-100 in PBS for 5 min. Cells were washed by PBS and then blocked by 5% BSA at room temperature for 1 h. Cells were incubated with mouse IgG anti-NP (Millipore, Cat #2817019, 1:600) at room temperature for 1 h and then washed by PBS for next incubation with goat anti-mouse IgG Alexa-488 (Life Technologies, Cat #1752514, 1:600) at room temperature for 1 h. Finally, cells were washed by PBS and stained with DAPI. Images were taken by confocal microscope (Carl Zeiss LSM 700, Germany).

### NMR Structure Analysis of P9R

Freshly prepared 1 mg ml⁻¹ (0.29 mM) of P9R in 0.5 ml solvent was used for the NMR study. Data were collected in H₂O/D₂O (19:1 v/v), as well as 99.996% D₂O, with the internal reference trimethylsilylpropanoic acid. All NMR spectra were acquired on either a Bruker AVANCE III 600 MHz spectrometer (Bruker BioSpin, Germany) or a Bruker AVANCE III 700 MHz spectrometer at 25° C. 2D ¹H-¹H correlation spectroscopy (COSY), total correlated spectroscopy (TOCSY) and nuclear Overhauser effect spectroscopy (NOESY) spectra were recorded for resonance assignments. Inter-proton distance restraints were derived from 2D NOESY spectrum with mixing times of 300 ms and 500 ms using automated NOE assignment strategy followed by a manual check. NOE intensities and chemical shifts were extracted using CCPNMR Analysis 2.4.2 (Skinner et al., J Biomol NMR 66, 111-124 (2016)) and served as inputs for the Aria program. Dihedral angle is predicted from the chemical shifts using the program DANGLE (Cheung et al., J Magn Reson 202, 223-233 (2010)). The NMR solution structure of P9R was calculated iteratively using Aria 2.3 program (Rieping et al., Bioinformatics 23, 381-382 (2007)). One hundred random conformers were annealed using distance restraints in each of the eight iteratively cycles of the combined automated NOE assignments and structure calculation algorithm. The final upper limit distance constraints output from the last iteration cycle were subjected to a thorough manual cross-checking and final water solvent structural refinement cycle. The 10 lowest energy conformers were retained from these refined 100 structures for statistical analysis. The convergence of the calculated structures was evaluated using root-mean-square deviations (RMSDs) analyses. The distributions of the backbone dihedral angles (φ, ψ) of the final converged structures were evaluated by representation of the Ramachandran dihedral pattern using PROCHECK-NMR (Laskowski et al., J Biomol NMR 8, 477-486 (1996)). Visualization of three-dimensional structures and electrostatic surface potential of P9R were achieved using UCSF Chimera 1.13.1 (Pettersen et al., J Comput Chem 25, 1605-1612 (2004)).

### Antiviral Analysis of P9R in Mice

BALB/c female mice, 10-12 weeks old, were kept in biosafety level 2 laboratory and given access to standard pellet feed and water ad libitum. All experimental protocols followed the standard operating procedures of the approved biosafety level 2 animal facilities and were approved by the Committee on the Use of Live Animals in Teaching and Research of the University of Hong Kong (Zheng et al., Proc Natl Acad Sci USA 105, 8091-8096 (2008)). The mouse adapted H1N1 virus was used for lethal challenge of mice. To evaluate the therapeutic effect, mice were challenged with 3 LD 50 of the virus and then intranasally inoculated with PBS, P9, P9R, PA1 or zanamivir at six hours after the viral inoculation. Two more doses were given to H1N1-challenged mice at the following one day. Survival and general conditions were monitored for 16 days or until death.

### Statistical Analysis

Survival of mice and the statistical significance were analyzed by GraphPad Prism 5. The statistical significance of the other results was calculated by the two-tailed Student t test using Stata statistical software. Results were considered significant at P<0.05.

### Results

Mouse β-Defensin-Derived Peptide P9R could Broadly Inhibit Coronaviruses and Other Respiratory Viruses

Endosomal acidification is affected by the influx of protons into the endosome via the vacuolar membrane proton pump V-ATPase (Huotari & Helenius, EMBO J 30, 3481-3500 (2011)). Theoretically, an alkaline peptide with stronger net positive charge would neutralize protons in the endosome, thereby inhibiting the endosomal acidification. Hence, to improve a previous antiviral peptide P9 (Zhao et al., Sci Rep 6, 22008 (2016)), the weakly positively charged amino acids (histidine and lysine) were substituted by arginine at positions 21 (H→R), 23 (K→R) and 28 (K→R) (FIG. 1A) to increase the net positive charge (+4.7) of P9 to charge (+5.6) of P9R. In the plaque reduction assay, the 1050 of P9R against SARS-CoV-2 was significantly lower than that of P9 (0.9 μg ml⁻¹ vs 2.4 μg ml⁻¹, P<0.01) (FIG. 1B). Furthermore, P9R showed significantly stronger inhibition against MERS-CoV, A(H1N1)pdm09 virus, A(H7N9) virus, and rhinovirus than P9 (FIGS. 1c-1g). However, the IC₅₀ of P9R and P9 against parainfluenza virus 3 was much higher (>25.0 m ml⁻¹), likely because endosomal acidification was not required in the viral life cycle of parainfluenza virus 3 (FIG. 1H) (Moscona, J Clin Invest 115, 1688-1698 (2005)). In the multicycle growth assay, P9R inhibited viral replication by 1000-fold for SARS-CoV-2, MFRS-CoV, and SARS-CoV (FIG. 1I). For A(H1N1)pdm09 virus, A(H7N9) virus and rhinovirus, P9R could inhibit >20-fold viral replication (FIG. 1I). In addition, the CC₅₀ of P9R was >300 μg ml⁻¹ for MDCK, VeroE6 and A549 cells (FIG. 1J). These results indicated that P9R with more positive charge could more efficiently inhibit the new coronavirus SARS-CoV-2 and other enveloped and non-enveloped respiratory viruses than that of P9.

The Degree of Positive Charge is Critical for the Inhibition of Endosomal Acidification and Antiviral Activity

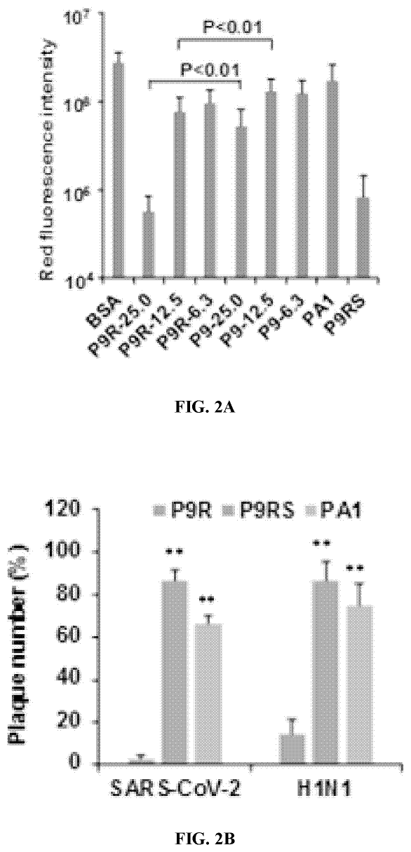

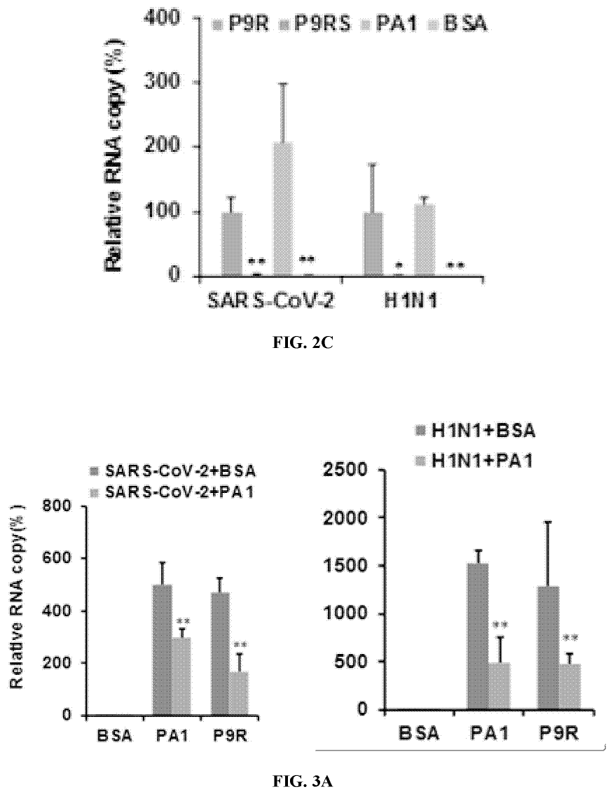

To determine whether the net charge of the peptide affects the inhibition of endosomal acidification, the endosomal acidification assay, identified that P9R (+5.6) could more significantly inhibit endosomal acidification in live cells than that of P9 (data nor shown, and FIG. 2A), which are consistent with the stronger antiviral activity of P9R than that of P9. In addition, peptide PA1 with less positive charge (+1.7), which has the same amino acid sequence as P9 except 3 additional acidic amino acid at the C terminal, could not inhibit endosomal acidification (data not shown and FIG. 2B) and lost the antiviral activity (FIG. 2B). Hence, the degree of net positive charge was correlated with the degree of inhibition of endosomal acidification and antiviral activity.

Inhibition of Host Endosomal Acidification Alone is not Sufficient for Positively Charged Peptide Inhibiting Virus Replication

To determine whether the antiviral activity solely relied on the positive charge of peptide, a peptide P9RS (+5.6) which had the same positive charge as P9R (+5.6) was designed, but P9RS differed from P9R by 11 of 30 amino acids. P9RS efficiently inhibited host endosomal acidification to the similar degree as P9R in live cells (data not shown and FIG. 2A). However, in the plaque reduction assay, there was no significant reduction of plaque numbers for SARS-CoV-2 and A(H1N1)pdm09 virus when viruses were treated by P9RS even at 25 μg ml⁻¹ (FIG. 2B).

To investigate why P9RS failed to inhibit viral replication despite potent inhibition of host endosomal acidification, the binding between the peptide and virus was studied. Using ELISA-RT-qPCR assay, P9R and PA1 could efficiently bind to SARS-CoV-2 and A(H1N1)pdm09 virus but P9RS did not bind to SARS-CoV-2 and A(H1N1)pdm09 virus (FIG. 2C). The observation of P9R but not P9RS binding to virus was further confirmed by confocal microscopy in H1N1-infected cells (data not shown). Thus, the direct interaction of peptide with virus was required for the antiviral activity of positively charged peptide P9R. In contrast, P9RS without the ability of binding to virus could not inhibit viral replication even though it carries the same positive charge as P9R and inhibits host endosomal acidification.

The Broad-Spectrum Antiviral Activity of P9R Relies on Targeting Viruses to Inhibit Virus-Host Endosome Acidification

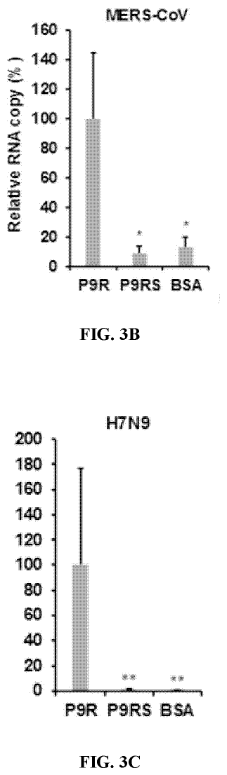

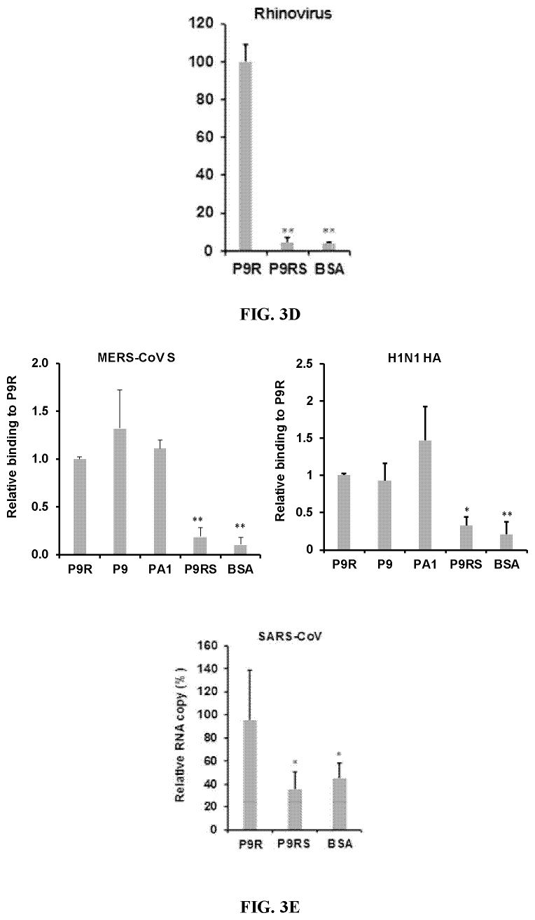

The above experiments, demonstrated that P9R and P9RS can inhibit no-virus endosomal acidification (data not shown and FIG. 2A). However, without binding to virus, P9RS could not inhibit viral replication. To illustrate this result, additional studies showed that P9R and bafilomycin A1 could efficiently inhibit the virus-host endosomal acidification in infected live cells, but P9RS could not inhibit the virus-host endosomal acidification in infected live cells (FIG. 3a), even though both of P9R and P9RS could inhibit the endosomal acidification of no-virus endosomes (data not shown). The efficient inhibition of P9R on virus-host endosomal acidification could be due to the binding of P9R to virus (data not shown and FIG. 2C) and then inhibiting the virus-host endosomal acidification (data not shown). Lacking the binding ability to viruses (data not shown and FIG. 2E), P9RS could not efficiently enter endosomes with the viruses to inhibit the virus-host endosomal acidification, possibly because the presence of virus in endosomes prevented the entry of unbonded P9RS into the endosomes. Without viruses in endosomes, there were empty spaces in no-virus endosomes to allow P9RS freely entering endosomes to prevent endosomal acidification (data not shown). It should be noted that PA1 with a similar sequence as P9R could efficiently bind to SARS-CoV-2 and A(H1N1)pdm09 virus (FIG. 2C), but it significantly lost the antiviral activity against SARS-CoV-2 and A(H1N1)pdm09 virus (FIG. 2B). The binding of P9R to SARS-CoV-2 and A(H1N1)pdm09 virus could be significantly reduced when viruses were pretreated by PA1 (FIG. 3A). This indicated that PA1 had the same binding sites on viral particles as P9R but only peptide binding to virus alone could not account for the antiviral activity. P9R binding to virus was the first step to exert the antiviral activity. After binding to virus (data not shown), P9R could efficiently inhibit virus-host endosomal acidification (Fidata not shown) and then inhibit viral replication by blocking RNP release (data not shown).

To further confirm that broad-spectrum antiviral activity of P9R was due to the broadly bindings of P9R to different viruses and viral proteins, additional studies demonstrated that P9R but not P9RS could also bind to MERS-CoV, A(H7N9) virus, rhinovirus, SARS-CoV and viral proteins (FIGS. 3B-3D and FIG. 3E). This result further confirmed that positively charged P9R could inhibit pH-dependent endosomal viruses if it can bind to viruses.

Next, studies were conducted to determine the structure of P9R using NMR spectroscopy. The results indicated that the solution structure of P9R was flexible with short variable helical patches and with positively charged peptide surface (data not shown). Without being bound by theory, P9R can broadly bind to different viruses because these short α-helical patches with flexible linkages may allow it to adapt its structure to fit the binding pockets of different viral proteins. In conclusion, the present studies demonstrate the novel antiviral mechanism that positively charged P9R needs to target viruses and then prevents virus-host endosomal acidification to inhibit pH-dependent virus replication.

The Efficacy of P9R Treatment In Vivo

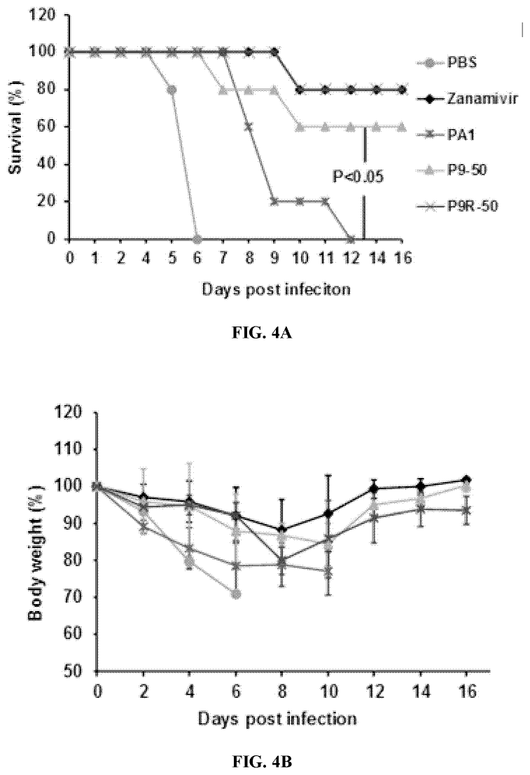

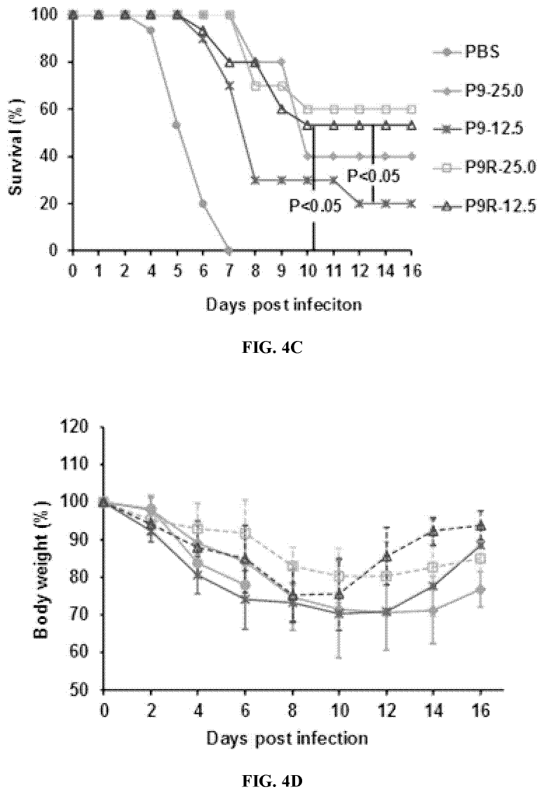

Studies demonstrated that the efficient antiviral activity of P9R in vitro is reliant on binding to viruses and the positive charge of P9R to inhibit virus-host endosomal acidification. To further investigate the antiviral activity of P9R in vivo, A(H1N1)pdm09-infected mice were treated at 6 h post infection with additional two doses in the following one day. In this model, 80% of P9R-treated mice survived, which was significantly better than PBS-treated group and PA1-treated group (FIG. 4A). The protection of P9R on infected mice was the same as that in the zanamivir-treated group (80%) and was better than P9. From day 4 to day 10 post infection, there was significantly less body weight loss in P9R group than that in PBS-treated group and PA1-treated group (FIG. 4B). The low dose protection of P9R (25 μg/dose and 12.5 μg/dose) on infected mice and reducing body weight loss further demonstrated that P9R could significantly protect mice when compared with PBS-treated group (FIGS. 4C and 4D). The antiviral activity of P9R in vivo was better than that of P9 (FIG. 4C, P<0.05 for 12.5 μg/dose), which was consistent with the significantly better antiviral activity of P9R than P9 in vitro.

No Emergence of Resistant Viruses Against P9R after Serial Passages of Virus in the Presence of P9R

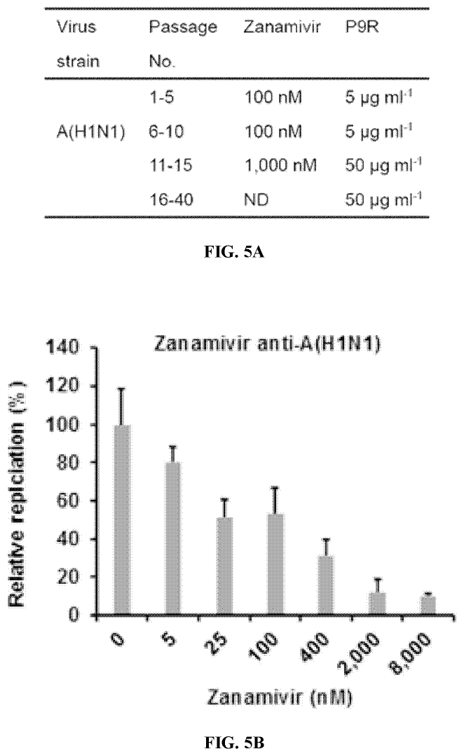

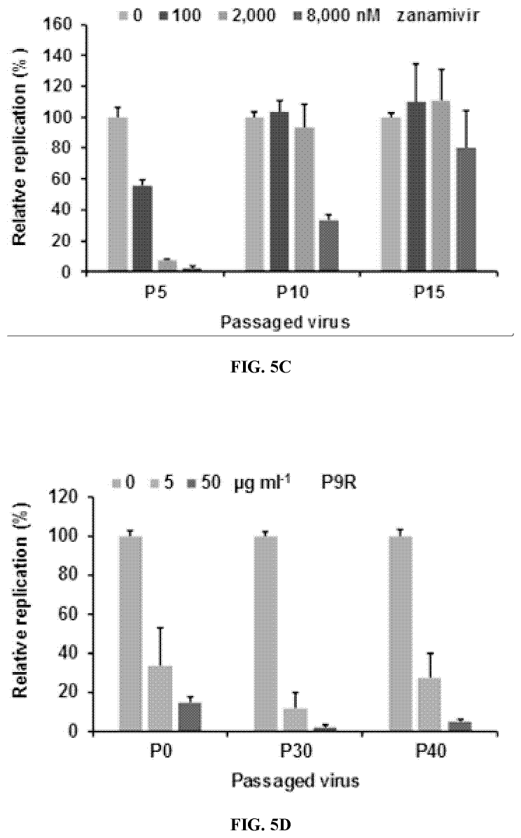

Emergence of resistant mutants occur from time to time (Zhao et al., Nat Commun 9, 2358 (2018)), especially with the new polymerase inhibitor baloxavir (Hayden et al., N Engl J Med 379, 913-923 (2018)). To determine whether P9R treatment induces viral resistance, A(H1N1)pdm09 virus was serially passaged 40 times in the presence of P9R in MDCK cells (FIG. 5A). A(H1N1)pdm09 virus was serially passaged in the presence of zanamivir as a control for resistance assay (FIG. 5A). The 1050 of zanamivir against parent A(H1N1)pdm09 virus (P0) was 35 nM (FIG. 5B). After 10-virus passages in the presence of zanamivir (100 nM) and additional 5-virus passages in the presence of zanamivir (1000 nM), 2000 nM and 8000 nM zanamivir could not inhibit P10 and P15 virus replication, respectively (FIG. 5C). These indicated that after 10 passages of virus in the presence of zanamivir had caused significant viral resistance to zanamivir. However, for P9R, even the A(H1N1)pdm09 virus was passaged in the presence (5.0 μg ml⁻¹ of P9R for the initial 10 passages and 50.0 μg ml⁻¹ for the rest 30 passages) of P9R for 40 passages, P9R (5.0 μg ml⁻¹) could efficiently inhibit P30 and P40 virus replication (FIG. 5D). No obvious drug-resistant virus to P9R was detected. These results indicated that P9R had very low possibility to cause drug-resistant virus.

### DISCUSSION

In this study, a broad-spectrum antiviral peptide P9R with potent antiviral activity against enveloped coronaviruses (SARS-CoV-2, SARS-CoV and MERS-CoV), influenza virus, and non-enveloped rhinovirus was identified. First, studies demonstrated that the antiviral activity of P9R could be significantly enhanced by increasing the net positive charge to more efficiently inhibit endosomal acidification. Second, mechanistic studies further demonstrated the novel antiviral mechanism that positively charged P9R could bind to different respiratory viruses to inhibit virus-host endosomal acidification. PA1 (only binding to viruses) or P9RS (only inhibiting endosomal acidification) did not show antiviral activity. Mechanistic studies showed that positively charged P9R broadly inhibits viral replication by binding to different viruses and then inhibiting virus-host endosomal acidification to prevent the endosomal release of pH-dependent viruses. P9R (not only binding to viruses but also inhibiting endosomal acidification), PA1 (only binding to viruses) and P9RS (only inhibiting endosomal acidification) were used to identify and confirm the novel antiviral mechanism of alkaline peptides. Third, the in vivo antiviral activity of P9R was demonstrated by protecting mice from lethal influenza virus challenge. The antiviral activity of alkaline peptide could be enhanced by increasing the positive charge of peptide and required both of binding to viruses and inhibiting endosomal acidification. Fourth, there was no reduced susceptibility of serial-passaged viruses (40 passages) against P9R.

Endosomal acidification is a key step in the life cycle of many pH-dependent viruses, which is one of the broad-spectrum antiviral targets (Vigant et al., Nat Rev Microbiol 13, 426-437 (2015)). In this study, with the increased positive charge in P9R, it could more efficiently inhibit pH-dependent viruses than P9. The more positive charge in P9R allowed the peptide to more efficiently neutralize protons inside endosomes, and thereby inhibiting the endosomal acidification. In previous studies, the clinically approved anti-malarial drug chloroquine with activity of inhibiting endosomal acidification had been demonstrated to inhibit enterovirus-A7 (Tan et al., Antiviral Res 149, 143-149 (2018)), zika virus (Li et al., EBioMedicine 24, 189-194 (2017)) and SARS-CoV-2 (Wang et al., Cell Res 30, 269-271 (2020)). The anti-parasitic drug niclosamide also inhibited influenza virus, rhinovirus, and dengue virus by interfering endosomal acidification (Jurgeit et al., PLoS Pathog 8, e1002976 (2012), Kao et al., PLoS Negl Trop Dis 12, e0006715 (2018)). However, researchers demonstrated the lack of protection of chloroquine in vivo for treating influenza virus and Ebola virus (Falzarano et al., Emerg Infect Dis 21, 1065-1067 (2015), Paton et al., Lancet Infect Dis 11, 677-683 (2011)). Differing from these drugs by interfering host endosomal acidification without targeting viruses, P9R inhibits viral replication by binding to viruses and then inhibiting virus-host endosomal acidification, which allows P9R to selectively and efficiently inhibit endosomal viruses. The protection of P9R on A(H1N1)-infected mice further confirmed the antiviral efficiency in vivo.

The antiviral activity of P9R required both of binding to viruses and inhibiting endosomal acidification. PA1 with less positive charge could not inhibit SARS-CoV-2 and H1N1 virus even though it had the similar binding ability and binding sites to viruses as P9R (FIG. 3b). When multiple substitutions were made on P9R to generate P9RS, P9RS lost the binding ability and antiviral activity to all tested viruses even though P9RS had the same positive charge as P9R and efficiently inhibited host endosomal acidification. While not being bound by theory, the broadly binding mechanism of P9R to different viral proteins may be due to the flexible structure of P9R with positively charged surface (FIG. 3h). The flexible structure may allow P9R to change its structure to fit targeting proteins for broad-specificity bindings (Seppala et al., PLoS One 10, e0136969 (2015), Nakano et al., Sci Rep 5, 13836 (2015)), and the positive charge of P9R may play roles for binding to viruses with negatively charged surface (Hammen et al., J Biol Chem 271, 21041-21048 (1996), Michen & Graule, J Appl Microbiol 109, 388-397 (2010)). The five cysteines in P9R may also affect the structure-based binding because previous studies indicated that cysteine substitutions could affect defensin-peptide structure and activity (Chandrababu et al., Biochemistry 48, 6052-6061 (2009), Liu et al., Chembiochem 9, 964-973 (2008)).

In addition, comparing with zanamivir which caused significant drug resistant virus after passages in the presence of zanamivir, P9R showed very low risk to cause drug-resistance virus even when A(H1N1)pdm09 virus was passaged in the presence of P9R for 40 passages.

In summary, most highly pathogenic emerging viruses are endosomal pH-dependent viruses. The emerging and re-emerging virus outbreaks remind us of the urgent need of broad-spectrum antivirals. The present studies provide one such broad-spectrum antiviral.

### Example 2: Multivalent P9R

### Materials and Methods

### Cells and Viruses

Madin Darby canine kidney (MDCK, CCL-34), Vero-E6 (CRL-1586), Calu-3 (HTB-LLC-MK2 (CCL-7) and RD (CCL136) cells obtained from ATCC (Manassas, VA, USA) were cultured in Dulbecco minimal essential medium (DMEM for Vero-E6 cells), MEM (for MDCK, LLC-MK2 and RD cells) or DMEM-F12 (for Calu-3 cells) supplemented with 10% fetal bovine serum (FBS), 100 NJ ml⁻¹ penicillin and 100 μg ml⁻¹ streptomycin. The virus strains used in this study included 2019 new coronavirus (SARS-CoV-2) (Chu, et al., Lancet Microbe 1, e14-e23 (2020)), SARS-CoV (Zhao, et al., Sci Rep 6, 22008 (2016)), A/Hong Kong/415742/2009 (Zhao, et al., Virology 498, 1-8 (2016)), human parainfluenza 3 (ATCC-C243) and clinical isolated rhinovirus.

### Plaque Reduction Assay

Peptides (P9R, P9RS and 8P9R) were synthesized by ChinaPeptide. Antiviral activity of peptides was measured using a plaque reduction assay. Briefly, peptides were dissolved in PBS or 30 mM phosphate buffer (PB) containing 24.6 mM Na₂HPO₄ and 5.6 mM KH₂PO₄ at a pH of 7.4. For the assay for coronavirus, peptides or bovine serum albumin (BSA, 0.2-25.0 μg ml⁻¹) were premixed with 50 PFU of coronavirus (SARS-CoV-2) in PBS or PB at room temperature. After 45-60 min of incubation, peptide-virus mixture was transferred to Vero-E6 cells, correspondingly. For the assay for influenza virus, A(H1N1)pdm09 virus was treated with 8P9R (25 μg/ml) or PBS (Mock) at room temperature for 45 min and then MDCK cells were infected with the treated virus. At 1 h post infection, infectious media were removed and 1% low melting agar was added to cells. Cells were fixed using 4% formalin at 3 day post infection. Crystal blue (0.1%) was added for staining, and the number of plaques was counted.

### Antiviral Multicycle Growth Assay

SARS-CoV-2 and SARS-CoV infected Vero-E6 (0.005 MOD or Calu-3 (0.05 MOO cells at the presence of drugs or with the supplemental drugs at indicated post infection time. After 1 h infection, infectious media were removed and fresh media with supplemental drugs were added to infected cells for virus culture. At 24 h post infection, the supernatants of infected cells were collected for plaque assay or RT-qPCR assay.

### Viral RNA Extraction and RT-qPCR

Viral RNA was extracted by Viral RNA Mini Kit (QIAGEN, Cat 52906, USA) according to the manufacturer's instructions. Extracted RNA was reverse transcribed to cDNA using PrimeScript II 1^{st} Strand cDNA synthesis Kit (Takara, Cat^{#} 6210A) using GeneAmp® PCR system 9700 (Applied Biosystems, USA). The cDNA was then amplified using specific primers (Table 2) for detecting SARS-CoV-2, SARS-CoV and parainfluenza virus 3 and rhinovirus using LightCycle® 480 SYBR Green I Master (Roach, USA). For quantitation, 10-fold serial dilutions of standard plasmid equivalent to 10¹ to 10⁶ copies per reaction were prepared to generate the calibration curve. Real-time qPCR experiments were performed using LightCycler® 96 system (Roche, USA).

TABLE 2
Primers

| | | Oligonucleotide |
|---|---|---|
| Gene | Primer | sequence (5′ to3′) |
| SARS- | S-F | CCTACTAAATTAAATGATCTCTGCT |
| CoV-2 | | TTACT (SEQ ID NO: 5) |
| | S-R | CAAGCTATAACGCAGCCTGTA |
| | | (SEQ ID NO: 6) |
| SARS- | NP-F | ACCAGAATGGAGGACGCAAT |
| CoV | | (SEQ ID NO: 9) |
| | NP-R | GCTGTGAACCAAGACGCAGTATTAT |
| | | (SEQ ID NO: 10) |
| parainfluenza | HPIV3_ | AGCTATYACTAGYATCTCAGGGT |
| virus 3 | Inner-F | (SEQ ID NO: 18) |
| | HPIV3_ | CCCAATCTGATCCACTGTGT |
| | Inner-Rm | (SEQ ID NO: 19) |
| Rhinovirus | 5′UTR-F | AGCCYGCGTGGCKGCC |
| | | (SEQ ID NO: 15) |
| | 5′UTR-R | AGCCYGCGTGGTGCCC |
| | | (SEQ ID NO: 16) |

### Hemolysis Assay

Two-fold diluted peptides in PBS were incubated with turkey red blood cells for 1 h at 37° C. PBS was used as a 0% lysis control and 0.1% Triton X-100 as 100% lysis control. Plates were centrifuged at 350 g for 3 min to pellet non-lysed red blood cells. Supernatants used to measure hemoglobin release were detected by absorbance at 450 nm (as discussed above).

### Cytotoxicity Assay

Cytotoxicity of peptides was determined by the detection of 50% cytotoxic concentration (CC₅₀) using a tetrazolium-based colorimetric MTT assay (Zhao, et al., Nat Commun 9, 2358 (2018)). Vero-E6 cells were seeded in 96-well cell culture plate at an initial density of 2×10⁴ cells per well in DMEM supplemented with 10% FBS and incubated for overnight. Cell culture media were removed and then DMEM supplemented with various concentrations of peptides and 1% FBS were added to each well. After 24 h incubation at 37° C., MTT solution (5 mg ml⁻¹, 10 μl per well) was added to each well for incubation at 37° C. for 4 h. Then, 100 μl of 10% SDS in 0.01M HCl was added to each well. After further incubation at room temperature with shaking overnight, the plates were read at OD570 using Victor™ X3 Multilabel Reader (PerkinElmer, USA). Cell culture wells without peptides were used as the experiment control and medium only served as a blank control.

### Transmission Electron Microscopy Assay

To determine the effect of 8P9R on viral particles, SARS-CoV-2 was pretreated by 50 μg ml⁻¹ of 8P9R, P9R or P9RS for 1 h. The virus was fixed by formalin for overnight and then applied to continuous carbon grids. The grids were transferred into 4% uranyl acetate and incubated for 1 min. After removing the solution, the grids were air-dried at room temperature. For each peptide/DNA nanoparticle, three independent experiments were done for taking images by transmission electron microscopy (FEI Tecnal G2-20 TEM).

### Virus Fluorescence Assay

To identify the effect of 8P9R on virus, H1N1 virus was pre-labelled by green Dio dye (Invitrogen, Cat #3898) according to the manufacture introduction. Dio-labeled virus was treated by 8P9R, P9RS, or P9R (25 μg ml⁻¹) for 45 min. MDCK cells were infected by the pretreated virus for 1 h. Virus and cells were fixed by 4% formalin. Cell membrane was stained by membrane dye Alexa 594 (red, Invitrogen, W11262) and cell nucleus were stained by DAPI (blue). Virus entry or without entry on cell membrane was determined by confocal microscope (Carl Zeiss LSM 700, Germany).

Endosomal Acidification Assay

Endosomal acidification was detected with a pH-sensitive dye (pHrodo Red dextran, Invitrogen, Cat #P10361) according to the manufacturer's instructions with slight modification (Zhao, et al., Nat Commun 9, 2358 (2018)). First, MDCK cells were treated with BSA (25.0 μg ml⁻¹), 8P9R (25.0 μg ml⁻¹), bafilomycin A1 (50.0 nM) at 4° C. for 15 min. Second, MDCK cells were added with 100 μg ml⁻¹ of pH-sensitive dye and DAPI and then incubated at 4° C. for 15 min. Before taking images, cells were further incubated at 37° C. for 15 min and then cells were washed twice with PBS. Finally, PBS was added to cells and images were taken immediately with confocal microscope (Carl Zeiss LSM 700, Germany).

### Spike-ACE2 Mediated Cell Fusion Assay

The pSpike of SARS-CoV-2, pACE2-human, or pGFP were transfected to 293T cells for protein expression. After 24 hours, to trigger the spike-ACE2 mediated cell fusion, 293T-Spike-GFP cell were co-cultured with 293T-ACE2 with the supplement of drugs. The 293T-GFP cells were co-cultured with 293T-ACE2 cells as the negative control. For Huh-7 cell fusion assay, Huh-7 cells were co-cultured with 293T-spike-GFP with the supplement of drugs. Huh-7 cells were co-cultured with 293T-GFP cells as the negative control. After 8 h of co-culture, five fields were randomly selected in each well to take the cell fusion pictures by fluorescence microscopes.

### Antiviral Assay in Animals

BALB/c female mice (10-month old) and hamsters (6-week old) were kept in biosafety level 2/3 laboratory (housing temperature between 22-25° C. with dark/light cycle) and given access to standard pellet feed and water ad libitum. All experimental protocols followed the standard operating procedures of the approved biosafety level 2/3 animal facilities. Animal ethical regulations were approved by the Committee on the Use of Live Animals in Teaching and Research of the University of Hong Kong (Zheng, et al., Proc Natl Acad Sci USA 105, 8091-8096 (2008)). To evaluate the antiviral activity, mice/hamsters were intranasally inoculated with SARS-CoV or SARS-CoV-2 to lungs. At 8 h post infection, PBS, 8P9R, arbidol, chloroquine, camostat, or combinational drugs were given to animals. Two more doses were given to mice/hamsters in the following one day. Viral loads in mouse/hamster lungs were measured at day 2 post infection by plaque assay.

### Results

8P9R Showed Potent Antiviral Activity Against SARS-CoV-2, H1N1, Parainfluenza Virus 3 and Human Rhinovirus

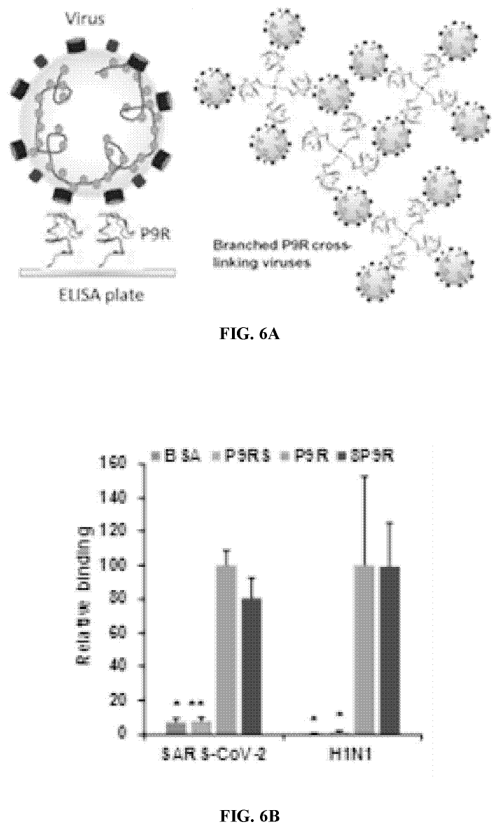

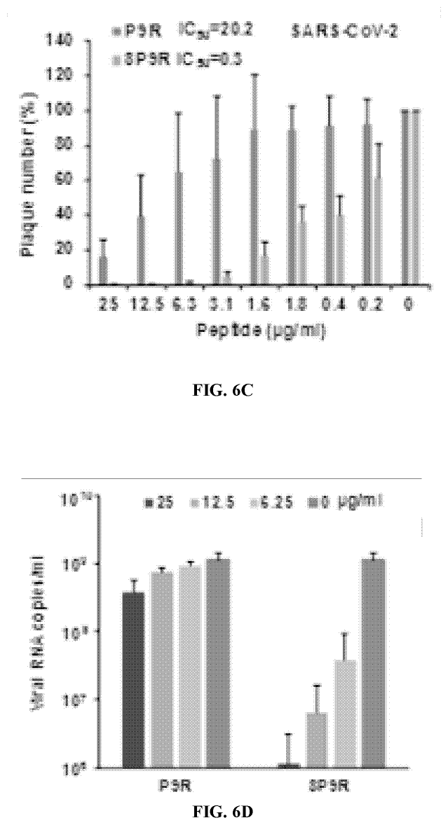

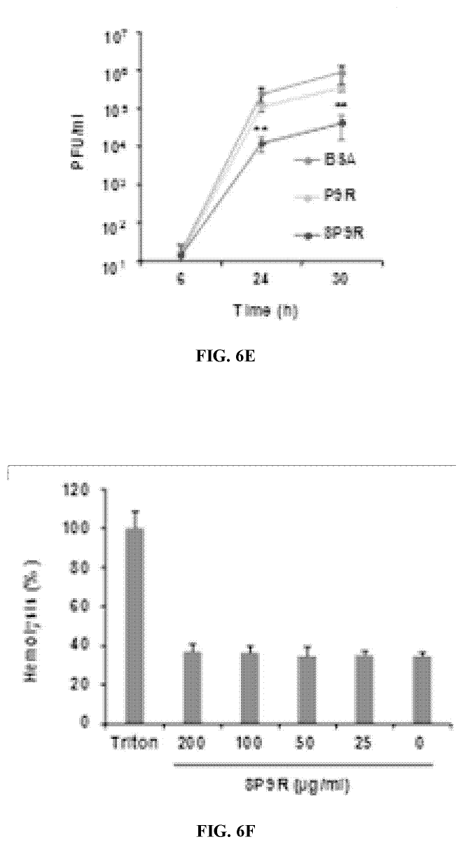

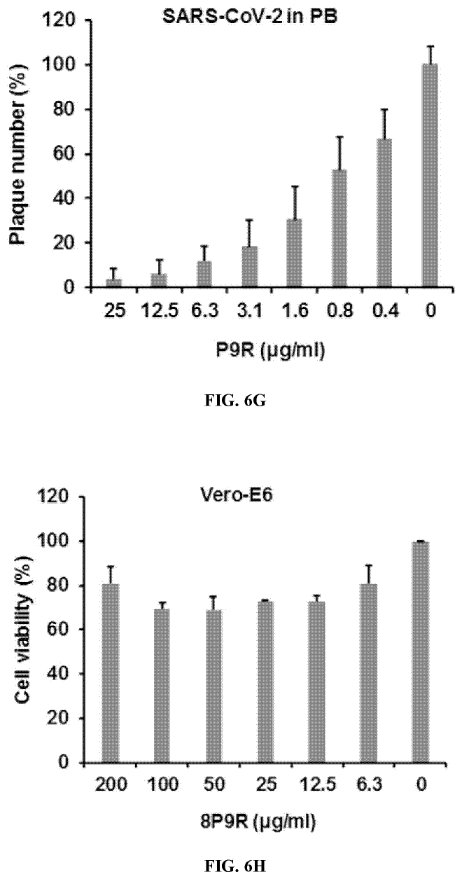

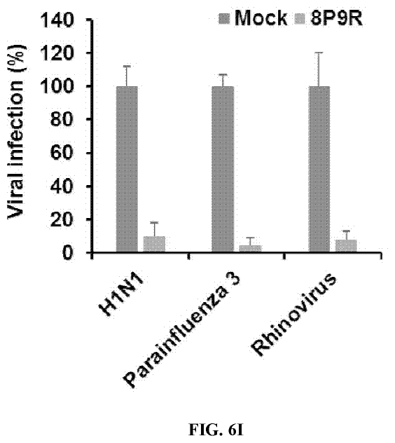

Example 1 shows that a broad-spectrum antiviral peptide P9R can suppress coronavirus and influenza virus by binding to viruses and inhibiting virus-host endosomal acidification. Experiments were designed to determine if a branched P9R could cross-link viruses (FIG. 6A) to enhance the antiviral activity. First, the binding ability of eight-branched P9R (8P9R) and single P9R to SARS-CoV-2 and H1N1 virus were determined by measuring the RNA copies of viruses binding to ELISA plate, on which peptides were coated. The viral RNA copies indicated that 8P9R could efficiently bind to viruses and capture viral particles on ELISA plate when compared with BSA and P9RS (FIG. 6B). This 8P9R suppressed SARS-CoV-2 infection more potently than P9R when viruses were pretreated by peptides (FIG. 6C), treated during viral inoculation (FIG. 6D) or post-infection (FIG. 6E). 8P9R showed more potent antiviral activity (IC₅₀=0.3 μg ml⁻¹) in high salt condition (PBS) than that (IC₅₀=20.2 μg ml⁻¹) of P9R in PBS (FIG. 6B), even though P9R showed potent antiviral activity (IC₅₀=0.9 μg ml⁻¹) in low salt concentration of 30 mM phosphate buffer (FIG. 6G). This is consistent with a previous report that antimicrobial activities of defensins are sensitive to high salt condition (Gong, et al., Arch Virol 155, 491-498 (2010)). Furthermore, no obvious hemolysis was observed when turkey red blood cells were treated by 8P9R at 200 μg ml⁻¹ (FIG. 6F) and the cytotoxicity assay indicated that TC₅₀ of 8P9R was higher than 200 μg ml⁻¹ in Vero-E6 cells (FIG. 6H). The viral infection (%) showed that 8P9R suppressed H1N1, parainfluenza virus 3 and human rhinovirus potently (FIG. 6I), indicating the broad-spectrum antiviral activity of 8P9R against respiratory viruses in addition to coronaviruses.

The Dual-Functional Activities of 8P9R Against Virus

To demonstrate the cross-linking ability, TEM images were taken to show that 8P9R could cross-link SARS-CoV-2 to form big viral cluster. In contrast, the peptide P9RS without binding ability (FIG. 6B) and single P9R did not cross-link virus to form big viral cluster. This was further confirmed this result with fluorescence-labelled H1N1 virus. The confocal pictures showed that 8P9R could efficiently cross-link H1N1 viruses that were aggregated around the cell membrane without entry when compared with the treatment of P9RS or P9R. Furthermore, it was demonstrated that 8P9R could efficiently inhibit endosomal acidification, which was similar to the endosomal acidification inhibitor bafilomycin A1. These results indicated the dual-functional activities of 8P9R which inhibited endosomal acidification required in endocytic pathway of viral infection and cross-linked viruses on the cell membrane surface without entry. The cross-linked viruses might affect SARS-CoV-2 entry on cell surface through TMPRSS2-mediated pathway. Thus, it was confirmed that 8P9R could inhibit SARS-CoV-2 infection through TMPRSS2-mediated surface entry pathway in Calu-3 cells in the later section.

8P9R could Enhance Arbidol at Low Concentration to Inhibit SARS-CoV-2

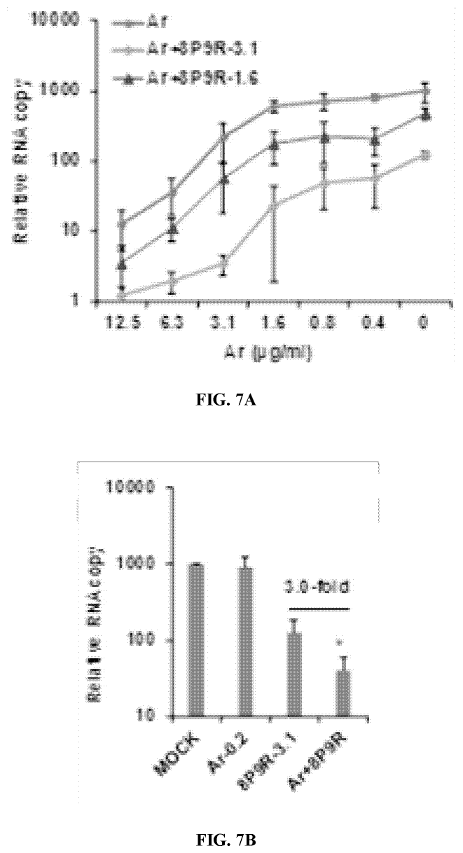

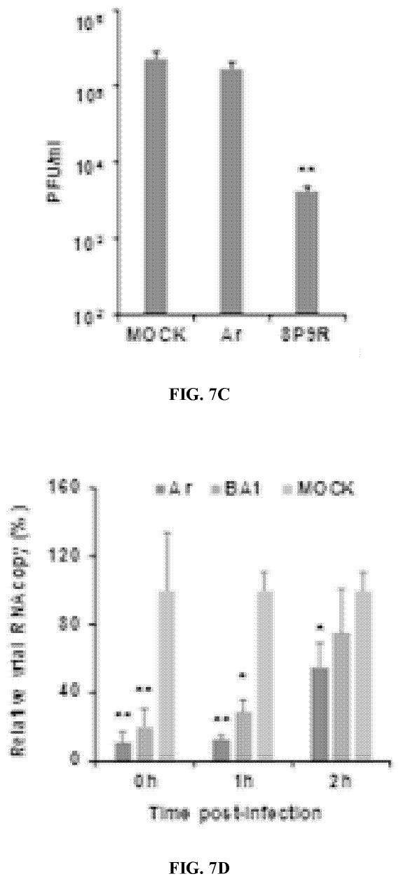

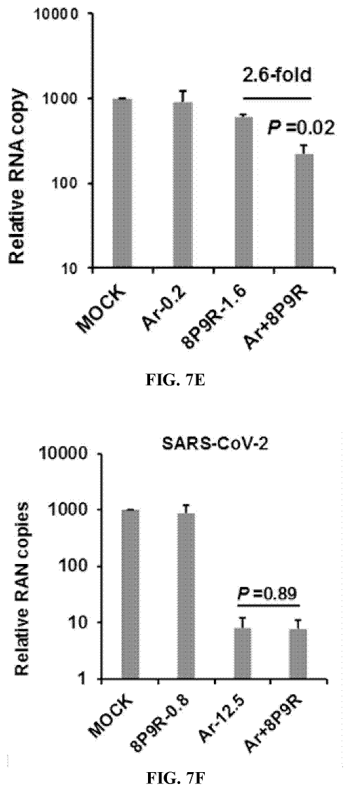

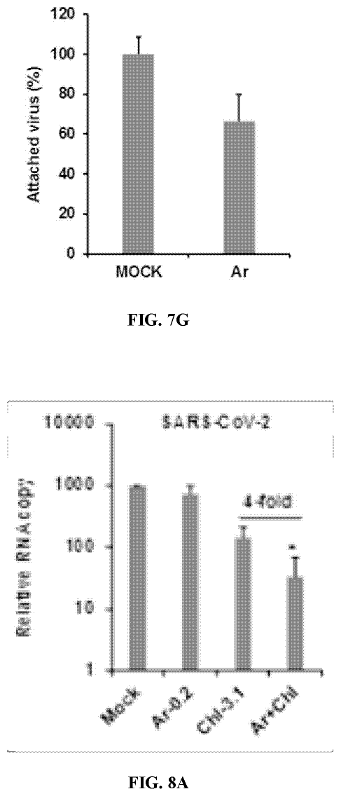

Serial monitoring by viral load and sequencing of clinical samples from COVID-19 patients showed that SARS-CoV-2 could be detected for more than one month with occasional detection of mutants (Koyama, et al., Bull World Health Organ 98, 495-504 (2020), Osman, Al Daajani, & Alsahafi, New Microbes New Infect 37, 100748 (2020)). These findings indicate potentially low sterilizing efficiency of human immune response for clearing SARS-CoV-2 in some patients. Thus, the repurposing of the anti-influenza drug arbidol available in China and Russia was considered. Arbidol showed in vitro antiviral activity against coronaviruses including SARS-CoV-2 and SARS-CoV. However, its relatively low serum concentration in human bodies (Deng, et al., Antimicrob Agents Chemother 57, 1743-1755 (2013), Sun, et al., Int J Clin Pharmacol Ther 51, 423-432 (2013)) may account for its poor antiviral efficacy in patients (Lian, et al., Clin Microbiol Infect 26, 917-921 (2020), Li, et al., Med (N Y) (2020)). Results showed that 8P9R significantly enhances the antiviral efficiency of arbidol at the concentration lower than the normal IC₅₀ (3.6 μg ml⁻¹) of arbidol (FIG. 7A). Importantly, 8P9R could elevate the antiviral activity of arbidol at low concentration (0.2 μg ml⁻¹) when arbidol itself did not show antiviral activity (FIG. 7B and FIG. 7E). This low concentration is closer or even lower than the concentration of arbidol in human serum. Furthermore, results proved that the synergistic activity was due to 8P9R enhancing arbidol, but not arbidol enhancing 8P9R (FIG. 7F), because arbidol (12.5 μg ml⁻¹) could not enhance 8P9R (0.8 μg ml⁻¹) to inhibit SARS-CoV-2 replication in Vero-E6 cells (FIG. 7F).

The Synergistic Mechanism of 8P9R Enhancing Arbidol Against SARS-CoV-2

To determine the synergistic enhancing mechanism of 8P9R on arbidol to inhibit SARS-CoV-2, arbidol's ability to slightly reduce viral attachment was first clarified (FIG. 7G). Next, when viruses (10⁶ PFU ml⁻¹) was pretreated by arbidol (25 μg ml⁻¹) and then diluted to folds for plaque assay, arbidol did not inhibit SARS-CoV-2 infection (FIG. 7C). In contrast, 8P9R could significantly reduce the number of infectious viruses even with >1,000-fold dilution, which indicated that the antiviral activity of 8P9R depended on targeting virus (FIG. 7C), similar to P9R (Zhao, et al., Nat Commun 11, 4252 (2020)). Results further showed that arbidol could significantly inhibit SARS-CoV-2 replication after viral entry in the time of addition experiment as that by bafilomycin A1, a known host targeting antiviral to inhibit cell endosomal acidification. (FIG. 7D). These results indicated that the main target of arbidol against SARS-CoV-2 is host cells, but not the virus. Next, arbidol was demonstrated to efficiently inhibit spike-ACE2 mediated cell-cell fusion in 293T cells (FIG. 7E) and Huh7 cells, which indicated that arbidol could inhibit virus-cell membrane fusion. The fusion inhibition of arbidol on SARS-CoV-2 was consistent with the claim that arbidol could block the release of SARS-CoV-2 in endolysosomes (Wang, et al., Cell Discov 6, 28 (2020)). Since lysosomes are the fusion location of SARS-CoV-2 infection through endocytic pathway (Li, Annu Rev Virol 3, 237-261 (2016)) and the endosomal acidification inhibitors, ammonium chloride (Hoffmann, et al., Cell 181, 271-280 e278 (2020)), bafilomycin A1 and 8P9R (125 μg ml⁻¹) could inhibit spike-ACE2 mediated cell membrane fusion (FIG. 7E), it was believed that the pH in endosomes/lysosomes could affect the inhibition efficiency of arbidol on spike-ACE2 mediated fusion. Using a low concentration of 8P9R combined with the low concentration of arbidol could more efficiently block the spike-ACE2-mediated membrane fusion (FIG. 7E) when compared with 8P9R or arbidol alone at 25 μg ml⁻¹. Thus, the mechanism of synergistic enhancement of arbidol by 8P9R but not 8P9R by arbidol is due to the inhibition of endosomal acidification by 8P9R, so that arbidol could more efficiently inhibit virus-cell fusion at the higher pH environment.

Endosomal Acidification Inhibitors Enhance Arbidol Against Coronaviruses

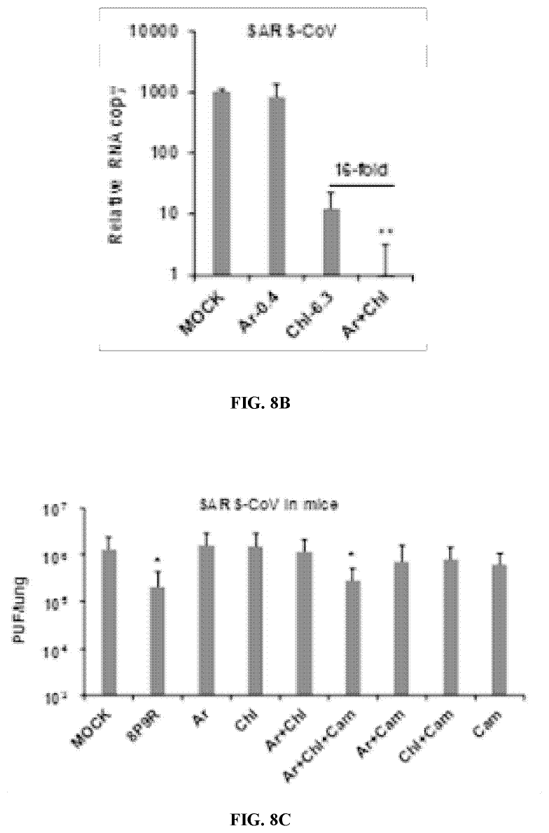

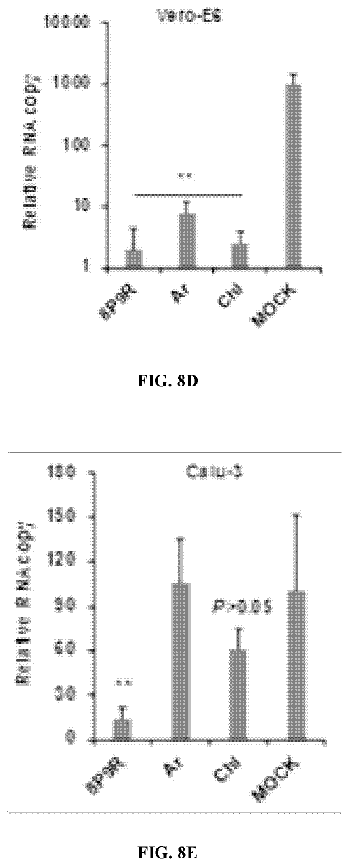

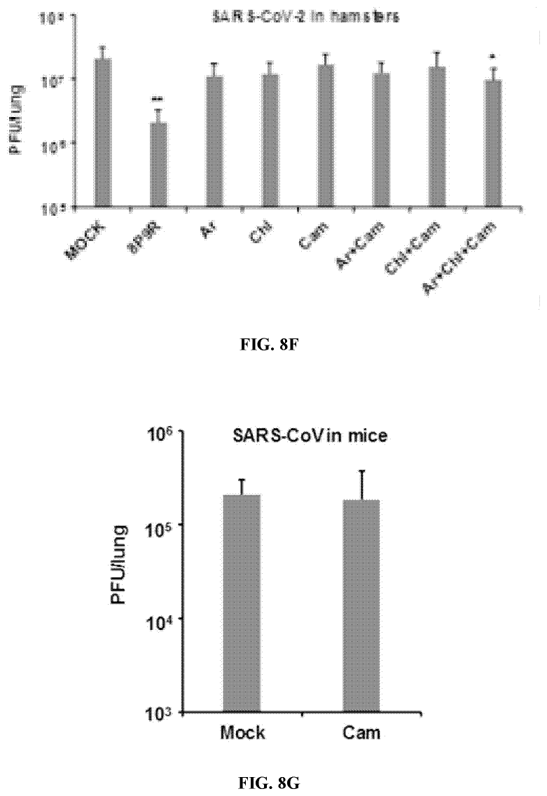

To further confirm the endosomal acidification inhibitors can synergistically enhance the antiviral activity of arbidol and to find clinically available drug for inhibiting SARS-CoV-2, it was identified that chloroquine, a known drug elevating endosomal pH, could significantly enhance the antiviral activity of arbidol at low concentrations (0.2-0.4 μg ml⁻¹) against SARS-CoV-2 (FIG. 8A) and SARS-CoV in Vero-E6 cells (FIG. 8B). Chloroquine supplemented with the low concentration of arbidol could inhibit more than 4-fold viral replication when compared with chloroquine alone (FIG. 8A-8B). The combination of chloroquine and arbidol could more effectively inhibit spike-ACE2 mediated cell-cell membrane fusion, which further confirmed that endosomal acidification inhibitors elevating pH in endosomes/lysosomes could enhance the antiviral activity of arbidol by blocking virus-cell membrane fusion. The findings support the combination of arbidol with chloroquine for better antiviral activity.

### Simultaneous Blockage of the Two Entry Pathways of Coronavirus for Antiviral Treatment In Vivo

To test the antiviral efficacy in vivo, 10-month-old mice were challenged with SARS-CoV and then drugs were initially administrated to mice at 8 h post infection. Arbidol (25 mg chloroquine (40 mg kg⁻¹) or the combination of arbidol with chloroquine could not inhibit SARS-CoV replication in mouse lungs (FIG. 8C). The dual-functional peptide 8P9R could significantly inhibit SARS-CoV replication in mouse lungs (FIG. 8C). This might indicate that inhibiting endocytic pathway of coronavirus infection alone could not efficiently inhibit coronavirus replication in vivo. Results showed (FIG. 8D), that arbidol and chloroquine could significantly inhibit SARS-CoV-2 replication in Vero-E6 cells (without TMPRSS2 (Matsuyama, et al., Proc Natl Acad Sci USA 117, 7001-7003 (2020))), but not in Calu-3 cells in which SARS-CoV-2 enters cells depending on TMPRSS2-mediated pathway (Hoffmann, et al., Nature (2020)) (FIG. 8E).

However, 8P9R could significantly inhibit SARS-CoV-2 in both Vero-E6 and Calu-3 cells (FIG. 8D-8E), which indicated that 8P9R not only inhibited the viral infection through endocytic pathway in Vero-E6 cells but also inhibited viral entry through TMPRSS2-mediated pathway in Calu-3 cells. The potent antiviral activity of 8P9R in Vero-E6, Calu-3 cells and in mouse model indicated that the simultaneous blockage of both entry pathways might more efficiently inhibit coronavirus replication in vivo. Camostat, a TMPRSS2 inhibitor, could significantly inhibit SARS-CoV-2 replication in Calu-3 cells (Hoffmann, et al., Cell 181, 271-280 e278 (2020)), but could not inhibit SARS-CoV-2 replication and pseudotyped particle entry in Vero-E6 cells (Hoffmann, et al., Cell 181, 271-280 e278 (2020), Hoffmann, et al., Nature (2020)). Thus, SARS-CoV-infected mice were treated with the combination of arbidol, chloroquine and camostat. This combination showed potent antiviral activity against SARS-CoV in mice (FIG. 8C), similar to the antiviral activity of 8P9R, whereas the drug combinations (arbidol and camostat or chloroquine and camostat) or camostat alone could not inhibit viral replication when compared with mock (FIG. 8C FIG. 8G). In parallel, this in vivo result was confirmed by treating SARS-CoV-2-infected hamsters with different drug combinations. Viral loads in hamster lungs showed that 8P9R or the triple combination of arbidol, chloroquine and camostat could significantly inhibit SARS-CoV-2 replication when compared with mock (FIG. 8F). Arbidol, chloroquine, or camostat alone, and camostat combined with chloroquine (FIG. 8F) could not significantly inhibit SARS-CoV-2 replication in hamsters. These findings confirmed the limited clinical efficacy of arbidol or chloroquine alone for treating SARS-CoV-2 in patients. More importantly, these results provided the evidences of using endosomal acidification inhibitors (8P9R or chloroquine) to enhance the antiviral activity of arbidol against SARS-CoV-2 infection through endocytic pathway. Moreover, dual-functional 8P9R or the triple drug combination of arbidol, chloroquine and camostat can effectively block the two entry pathways of coronavirus, which translates into significant reduction of viral replication in vivo.

### DISCUSSION

In this study, a dual-functional antiviral peptide 8P9R was developed which could cross-link viruses to block viral entry on cell surface through the TMPRSS2-mediated pathway and simultaneously inhibited endosomal acidification to block viral entry through endocytic pathway. The synergistic antiviral mechanism of endosomal acidification inhibitors (8P9R and chloroquine) on enhancing the activity of arbidol against SARS-CoV-2 and SARS-CoV infection through the endocytic pathway was demonstrated. Moreover, the triple combination of arbidol, chloroquine and camostat, which are currently available clinical drugs, was demonstrated for the suppression of SARS-CoV-2 replication in hamsters and SARS-CoV in mice. Both the triple drug combination and 8P9R could significantly inhibit SARS-CoV-2 and SARS-CoV in vivo, which indicated that blocking the two entry pathways of coronavirus infection is a promising approach for treating COVID-19.

SARS-CoV-2 and SARS-CoV can infect host cells by either TMPRSS2-mediated pathway or endocytic pathway. Recent studies indicated that chloroquine did not inhibit SARS-CoV-2 replication in Calu-3 cells (Hoffmann, et al., Nature (2020)) and camostat did not inhibit SARS-CoV-2 replication in Vero-E6 cells (Hoffmann, et al., Cell 181, 271-280 e278 (2020), Hoffmann, et al., Nature (2020)). By using a multi-targeting drug or drug combination to block the two entry pathways of coronavirus infection might be more efficient in inhibiting viral replication in patients because different human cells could express ACE2 and TMPRSS2 separately or simultaneously (Sungnak, et al., Nat Med 26, 681-687 (2020)). Endosomal acidification inhibitors (chloroquine and 8P9R) were shown to synergistically enhance the antiviral activity of arbidol against SARS-CoV-2 and SARS-CoV. It is believed that endosomal acidification inhibitors, by elevating endosomal pH, could enhance the activity of arbidol in blocking the spike-ACE2-mediated membrane fusion (FIG. 7E), which was consistent with the finding that spike-ACE2-mediated pseudotyped-particle entry was significantly affected by pH (ammonium chloride) in 293T cells (Hoffmann, et al., Cell 181, 271-280 e278 (2020)). However, the combination of chloroquine with arbidol did not show antiviral activity against SARS-CoV-2 and SARS-CoV in hamsters and mice. The possible reason is that chloroquine and arbidol can only inhibit SARS-CoV-2 replication by interfering with the endocytic pathway, but not the TMPRSS2-mediated pathway (FIG. 8D-8E). In contrast, 8P9R could significantly inhibit coronaviruses in vivo. 8P9R not only blocked the endocytic pathway by preventing endosomal acidification, but also cross-linked viral particles on cell membrane to reduce viral entry through the TMPRSS2-mediated pathway. The combination of chloroquine and camostat could not significantly inhibit both viruses in vivo, which is probably due to the marginal antiviral activity of chloroquine on inhibiting viral infection through endocytic pathway in mice, hamsters and ferrets (Falzarano, et al., Emerg Infect Dis 21, 1065-1067 (2015), Vigerust, et al., Influenza Other Respir Viruses, 1, 189-192 (2007)). The combination of arbidol with chloroquine could more efficiently inhibit viral infection through endocytic pathway in TMPRSS2-deficient Vero-E6 cells (FIG. 8A-8B). Thus, the triple combination of arbidol, chloroquine and camostat could significantly inhibit both SARS-CoV-2 and SARS-CoV replication in hamsters and mice (FIG. 8D) through simultaneous blockage of both entry pathways. Furthermore, these drugs are harnessing the host factors to interfere with viral replication which may therefore be less prone to induce drug resistant viral mutants.

With the widespread circulation of SARS-CoV-2 during the COVID-19 pandemic, the emergence of virus mutants and the decreasing antibody titers after recovery should alert us to the possibility of re-infection. The development of broad-spectrum antivirals is urgently needed for SARS-CoV-2 and new emerging viruses. Here, the antiviral peptide 8P9R was identified with dual functions to inhibit viral infection by cross-linking viruses to reduce viral entry on cell surface (ie. TMPRSS2-mediated entry pathway for SARS-CoV) and by interfering endosomal acidification to block viral entry through endocytic pathway. Furthermore, the data supported the use of combination drug treatment with currently available broad-spectrum drugs (arbidol, chloroquine and camostat) to block both entry pathways of SARS-CoV-2, which could be also the potential therapeutics for other respiratory viruses.

The disclosed compositions, and methods can be further understood through the following numbered paragraphs.

1. An antiviral agent comprising a multivalent peptide, wherein the multivalent peptide comprises three or more copies of one or a combination of peptides selected from the group consisting of P9R (SEQ ID NO:2) and P9R-like peptides derived from P9R, wherein at least three of the peptides that comprise the multivalent peptide branch from one or more of the peptides that comprise the multivalent peptide.

2. The antiviral agent of paragraph 1, wherein the multivalent peptide comprises six or more copies of the peptides, wherein at least six of the peptides that comprise the multivalent peptide branch from one or more of the peptides that comprise the multivalent peptide.

3. The antiviral agent of paragraph 1 or 2, wherein the multivalent peptide comprises eight or more copies of the peptides, wherein at least eight of the peptides that comprise the multivalent peptide branch from one or more of the peptides that comprise the multivalent peptide.

4. The antiviral agent of any one of paragraphs 1-3, wherein at least three of the peptides that branch from one or more of the peptides that comprise the multivalent peptide branch from a central point in the multivalent peptide.

5. The antiviral agent of any one of paragraphs 1-4, wherein at least six of the peptides that branch from one or more of the peptides that comprise the multivalent peptide branch from a central point in the multivalent peptide.

6. The antiviral agent of any one of paragraphs 1-5, wherein at least eight of the peptides that branch from one or more of the peptides that comprise the multivalent peptide branch from a central point in the multivalent peptide.

7. The antiviral agent of any one of paragraphs 1-6, wherein the peptides that branch from one or more of the peptides that comprise the multivalent peptide branch from a central point in the multivalent peptide.

8. The antiviral agent of any one of paragraphs 1-7, wherein the P9R-like peptides are characterized in that the P9R-like peptide inhibits endosomal acidification and retains virus binding as determined by an in vitro endosomal acidification, optionally compared to a control, and a peptide-virus binding assays.

9. The antiviral agent of any one of paragraphs 1-8, wherein the peptides that comprise the multivalent peptide each consist of P9R (SEQ ID NO:2).

10. The antiviral agent of any one of paragraphs 1-9, wherein one or more of the peptides that comprise the multivalent peptide has a net positive charge of at least 5.

11. The antiviral agent of any one of paragraphs 1-10, wherein the peptides that comprise the multivalent peptide each has a net positive charge of at least 5.

12. The antiviral agent of any one of paragraphs 1-11, wherein one or more of the peptides that comprise the multivalent peptide has a net positive charge of about 5.6.

13. The antiviral agent of any one of paragraphs 1-12, wherein the peptides that comprise the multivalent peptide each has a net positive charge of about 5.6.

14. The antiviral agent of any one of paragraphs 1-13, wherein one or more of the peptides that comprise the multivalent peptide has a net positive charge of 5.6.

15. The antiviral agent of any one of paragraphs 1-14, wherein the peptides that comprise the multivalent peptide each has a net positive charge of 5.6.

16. An antiviral agent comprising P9R (SEQ ID NO:2), or a P9R-like peptides derived from P9R.

17. The antiviral agent of paragraph 16, wherein the P9R-like peptide is characterized in that the P9R-like peptide inhibits endosomal acidification and retains virus binding as determined by an in vitro endosomal acidification, optionally compared to a control, and a peptide-virus binding assays.

18. The antiviral agent of paragraph 16 or 17, consisting of P9R (SEQ ID NO:2).

19. The antiviral agent of any one of paragraphs 16-18, wherein the antiviral agent has a net positive charge of at least 5.

20. The antiviral agent of any one of paragraphs 16-19, wherein the antiviral agent has a net positive charge of about 5.6.

21. The antiviral agent of any one of paragraphs 16-20, wherein the antiviral agent has a net positive charge of 5.6.

22. A composition comprising a therapeutically effective amount of the antiviral agent of any one of paragraphs 1-21 and a pharmaceutically acceptable carrier.

23. An antiviral composition comprising arbidol, chloroquine, and camostat.

24. The composition of paragraph 22 or 23, wherein the composition inhibits antiviral replication in the subject.

25. The composition of any one of paragraphs 22-24, wherein the composition is a unit dosage form.

26. The composition of paragraph 25, wherein the unit dosage form is selected from the group consisting of a table or capsule.

27. The composition of any one of paragraphs 22-24, in a form suitable for intranasal or pulmonary delivery.

28. The composition of paragraph 25, wherein the unit dosage form is an injectable, wherein the composition further comprises a pharmaceutically acceptable carrier for injection to a human.

29. A method of treating a viral infection in a subject in need thereof, the method comprising administering an effective amount of the antiviral agent of any one of paragraphs 1-21 or the composition of any one of paragraphs 22-28, to the subject.

30. The method of paragraph 29, wherein the infection is caused by a respiratory virus.

31. The method of paragraph 29 or 30, wherein the infection is caused by a pH-dependent virus that requires endosomal acidification for virus-host membrane fusion

32. The method of any one of paragraphs 29-31, wherein the composition is administered parenterally or orally.

33. The method of any one of paragraphs 29-31, wherein the composition is administered intranasally, or by pulmonary administration.

34. The method of paragraph of any one of paragraphs 29-33, wherein the infection is caused by zika virus, enterovirus-A7, ebola virus, influenza virus, SARS-CoV-2, SARS-CoV, MERS-CoV, the A(H1N1)pdm09 virus, avian influenza A(H7N9) virus, and the non-enveloped rhinovirus.

Those skilled in the art will recognize, or be able to ascertain using no more than routine experimentation, many equivalents to the specific embodiments of the invention described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different moieties does not indicate that the listed moieties are obvious one to the other, nor is it an admission of equivalence or obviousness.

### REFERENCES

Arvin, A. M. et al. A perspective on potential antibody-dependent enhancement of SARS-CoV-2. Nature 584, 353-363 (2020).
Borba, M. G. S. et al. Effect of High vs Low Doses of Chloroquine Diphosphate as Adjunctive Therapy for Patients Hospitalized With Severe Acute Respiratory Syndrome Coronavirus 2 (SARS-CoV-2) Infection: A Randomized Clinical Trial. JAMA Netw Open 3, e208857 (2020).
Brice, D. C. & Diamond, G. Antiviral Activities of Human Host Defense Peptides. Curr Med Chem (2019).
Chan, J. F. et al. A familial cluster of pneumonia associated with the 2019 novel coronavirus indicating person-to-person transmission: a study of a family cluster. Lancet 395, 514-523 (2020).
Chan, J. F. et al. Middle East respiratory syndrome coronavirus: another zoonotic betacoronavirus causing SARS-like disease. Clinical microbiology reviews 28, 465-522 (2015).
Chandrababu, K. B., Ho, B. & Yang, D. Structure, dynamics, and activity of an all-cysteine mutated human beta defensin-3 peptide analogue. Biochemistry 48, 6052-6061 (2009).
Cheng, V. C., To, K. K., Tse, H., Hung, I. F. & Yuen, K. Y. Two years after pandemic influenza A/2009/H1N1: what have we learned? Clinical microbiology reviews 25, 223-263 (2012).
Cheung, M. S., Maguire, M. L., Stevens, T. J. & Broadhurst, R. W. DANGLE: A Bayesian inferential method for predicting protein backbone dihedral angles and secondary structure. J Magn Reson 202, 223-233 (2010).
Chu, H. et al. Comparative tropism, replication kinetics, and cell damage profiling of SARS-CoV-2 and SARS-CoV with implications for clinical manifestations, transmissibility, and laboratory studies of COVID-19: an observational study. Lancet Microbe 1, e14-e23 (2020).
Deng, P. et al. Pharmacokinetics, metabolism, and excretion of the antiviral drug arbidol in humans. Antimicrob Agents Chemother 57, 1743-1755 (2013).
Dong, L., Hu, S. & Gao, J. Discovering drugs to treat coronavirus disease 2019 (COVID-19). Drug Discov Ther 14, 58-60 (2020).
Erickson, T. B., Chai, P. R. & Boyer, E. W. Chloroquine, hydroxychloroquine and COVID-19. Toxicol Commun 4, 40-42 (2020).
Falzarano, D. et al. Lack of protection against ebola virus from chloroquine in mice and hamsters. Emerg Infect Dis 21, 1065-1067 (2015).
Gomes, B. et al. Designing improved active peptides for therapeutic approaches against infectious diseases. Biotechnol Adv 36, 415-429 (2018).
Gong, T. et al. Recombinant mouse beta-defensin 2 inhibits infection by influenza A virus by blocking its entry. Arch Virol 155, 491-498 (2010).
Gordon, D. E. et al. A SARS-CoV-2 protein interaction map reveals targets for drug repurposing. Nature 583, 459-468 (2020).
Guo, X. et al. Longer Duration of SARS-CoV-2 Infection in a Case of Mild COVID-19 With Weak Production of the Specific IgM and IgG Antibodies. Front Immunol 11, 1936 (2020).
Hammen, P. K., Waltner, M., Hahnemann, B., Heard, T. S. & Weiner, H. The role of positive charges and structural segments in the presequence of rat liver aldehyde dehydrogenase in import into mitochondria. J Biol Chem 271, 21041-21048 (1996).
Hashem, A. M. et al. Therapeutic use of chloroquine and hydroxychloroquine in COVID-19 and other viral infections: A narrative review. Travel Med Infect Dis 35, 101735 (2020).
Hayden, F. G. et al. Baloxavir Marboxil for Uncomplicated Influenza in Adults and Adolescents. N Engl J Med 379, 913-923 (2018).
Hoffmann, M. et al. Chloroquine does not inhibit infection of human lung cells with SARS-CoV-2. Nature (2020).
Hoffmann, M. et al. SARS-CoV-2 Cell Entry Depends on ACE2 and TMPRSS2 and Is Blocked by a Clinically Proven Protease Inhibitor. Cell 181, 271-280 e278 (2020).
Hulseberg, C. E. et al. Arbidol and Other Low-Molecular-Weight Drugs That Inhibit Lassa and Ebola Viruses. Journal of virology 93 (2019).
Huotari, J. & Helenius, A. Endosome maturation. EMBO J 30, 3481-3500 (2011).
Ibarrondo, F. J. et al. Rapid Decay of Anti-SARS-CoV-2 Antibodies in Persons with Mild Covid-19. N Engl J Med 383, 1085-1087 (2020).
Jurgeit, A. et al. Niclosamide is a proton carrier and targets acidic endosomes with broad antiviral effects. PLoS Pathog 8, e1002976 (2012).
Kadam, R. U. & Wilson, I. A. Structural basis of influenza virus fusion inhibition by the antiviral drug Arbidol. Proc Natl Acad Sci USA 114, 206-214 (2017).
Kao, J. C. et al. The antiparasitic drug niclosamide inhibits dengue virus infection by interfering with endosomal acidification independent of mTOR. PLoS Negl Trop Dis 12, e0006715 (2018).
Klotman, M. E. & Chang, T. L. Defensins in innate antiviral immunity. Nat Rev Immunol 6, 447-456 (2006).
Koyama, T., Platt, D. & Parida, L. Variant analysis of SARS-CoV-2 genomes. Bull World Health Organ 98, 495-504 (2020).
Laskowski, R. A., Rullmannn, J. A., MacArthur, M. W., Kaptein, R. & Thornton, J. M. AQUA and PROCHECK-NMR: programs for checking the quality of protein structures solved by NMR. J Biomol NMR 8, 477-486 (1996).
Lau, S. K. et al. Severe acute respiratory syndrome coronavirus-like virus in Chinese horseshoe bats. Proc Natl Acad Sci USA 102, 14040-14045 (2005).
Lee, W. S., Wheatley, A. K., Kent, S. J. & DeKosky, B. J. Antibody-dependent enhancement and SARS-CoV-2 vaccines and therapies. Nat Microbiol (2020).
Leikina, E. et al. Carbohydrate-binding molecules inhibit viral fusion and entry by crosslinking membrane glycoproteins. Nat Immunol 6, 995-1001 (2005).
Li, C. et al. Chloroquine, a FDA-approved Drug, Prevents Zika Virus Infection and its Associated Congenital Microcephaly in Mice. EBioMedicine 24, 189-194 (2017).
Li, F. et al. A scorpion venom peptide Ev37 restricts viral late entry by alkalizing acidic organelles. J Biol Chem 294, 182-194 (2019).
Li, F. Structure, Function, and Evolution of Coronavirus Spike Proteins. Annu Rev Virol 3, 237-261 (2016).
Li, Q. et al. Early Transmission Dynamics in Wuhan, China, of Novel Coronavirus-Infected Pneumonia. N Engl J Med (2020).
Li, Q. et al. Virucidal activity of a scorpion venom peptide variant mucroporin-Ml against measles, SARS-CoV and influenza H5N1 viruses. Peptides 32, 1518-1525 (2011).
Li, Y. et al. Efficacy and Safety of Lopinavir/Ritonavir or Arbidol in Adult Patients with Mild/Moderate COVID-19: An Exploratory Randomized Controlled Trial. Med (N Y) (2020).
Lian, N. et al. Umifenovir treatment is not associated with improved outcomes in patients with coronavirus disease 2019: a retrospective study. Clin Microbiol Infect 26, 917-921 (2020).
Liu, A. et al. Disappearance of antibodies to SARS-CoV-2 in a -COVID-19 patient after recovery. Clin Microbiol Infect (2020).
Liu, S. et al. Different from the HIV fusion inhibitor C34, the anti-HIV drug Fuzeon (T-20) inhibits HIV-1 entry by targeting multiple sites in gp41 and gp120. J Biol Chem 280, 11259-11273 (2005).
Liu, S. et al. Linear analogues of human beta-defensin 3: concepts for design of antimicrobial peptides with reduced cytotoxicity to mammalian cells. Chembiochem 9, 964-973 (2008).
Long, Q. X. et al. Clinical and immunological assessment of asymptomatic SARS-CoV-2 infections. Nat Med 26, 1200-1204 (2020).
Lu, L. et al. Structure-based discovery of Middle East respiratory syndrome coronavirus fusion inhibitor. Nat Commun 5, 3067 (2014).
Maisonnasse, P. et al. Hydroxychloroquine use against SARS-CoV-2 infection in non-human primates. Nature (2020).
Matsuyama, S. et al. Enhanced isolation of SARS-CoV-2 by TMPRSS2-expressing cells. Proc Natl Acad Sci USA 117, 7001-7003 (2020).
Michen, B. & Graule, T. Isoelectric points of viruses. J Appl Microbiol 109, 388-397 (2010).
Moscona, A. Entry of parainfluenza virus into cells as a target for interrupting childhood respiratory disease. J Clin Invest 115, 1688-1698 (2005).
Nakano, S. et al. Structural and computational analysis of peptide recognition mechanism of class-C type penicillin binding protein, alkaline D-peptidase from Bacillus cereus DF4-B. Sci Rep 5, 13836 (2015).
Osman, A. A., Al Daajani, M. M. & Alsahafi, A. J. Re-positive coronavirus disease 2019 PCR test: could it be a reinfection? New Microbes New Infect 37, 100748 (2020).
Paton, N. I. et al. Chloroquine for influenza prevention: a randomised, double-blind, placebo controlled trial. Lancet Infect Dis 11, 677-683 (2011).
Pettersen, E. F. et al. UCSF Chimera—a visualization system for exploratory research and analysis. J Comput Chem 25, 1605-1612 (2004).
Rajendran, L., Knolker, H. J. & Simons, K. Subcellular targeting strategies for drug design and delivery. Nat Rev Drug Discov 9, 29-42 (2010).
Rebeaud, M. E. & Zores, F. SARS-CoV-2 and the Use of Chloroquine as an Antiviral Treatment. Front Med (Lausanne) 7, 184 (2020).
Rieping, W. et al. ARIA2: automated NOE assignment and data integration in NMR structure calculation. Bioinformatics 23, 381-382 (2007).
Riva, L. et al. Discovery of SARS-CoV-2 antiviral drugs through large-scale compound repurposing. Nature (2020).
Sample, C. J. et al. A mastoparan-derived peptide has broad-spectrum antiviral activity against enveloped viruses. Peptides 48, 96-105 (2013).
Sariol, A. & Perlman, S. Lessons for COVID-19 Immunity from Other Coronavirus Infections. Immunity 53, 248-263 (2020).
Seppala, J. et al. Flexible Structure of Peptide-Bound Filamin A Mechanosensor Domain Pair 20-21. PLoS One 10, e0136969 (2015).
Skinner, S. P. et al. CcpNmr AnalysisAssign: a flexible platform for integrated NMR analysis. J Biomol NMR 66, 111-124 (2016).
Smith, J. G. & Nemerow, G. R. Mechanism of adenovirus neutralization by Human alpha-defensins. Cell Host Microbe 3, 11-19 (2008).
Sun, Y. et al. Pharmacokinetics of single and multiple oral doses of arbidol in healthy Chinese volunteers. Int J Clin Pharmacol Ther 51, 423-432 (2013).
Sungnak, W. et al. SARS-CoV-2 entry factors are highly expressed in nasal epithelial cells together with innate immune genes. Nat Med 26, 681-687 (2020).
Swanson, M. D. et al. Engineering a therapeutic lectin by uncoupling mitogenicity from antiviral activity. Cell 163, 746-758 (2015).
Tan, Y. W., Yam, W. K., Sun, J. & Chu, J. J. H. An evaluation of Chloroquine as a broad-acting antiviral against Hand, Foot and Mouth Disease. Antiviral Res 149, 143-149 (2018).
To, K. K. et al. Consistent detection of 2019 novel coronavirus in saliva. Clin Infect Dis (2020).
To, K. K. et al. COVID-19 re-infection by a phylogenetically distinct SARS-coronavirus-2 strain confirmed by whole genome sequencing. Clin Infect Dis (2020).
To, K. K. et al. Pulmonary and extrapulmonary complications of human rhinovirus infection in critically ill patients. J Clin Virol 77, 85-91 (2016).
To, K. K., Chan, J. F., Chen, H., Li, L. & Yuen, K. Y. The emergence of influenza A H7N9 in human beings 16 years after influenza A H5N1: a tale of two cities. Lancet Infect Dis 13, 809-821 (2013).
Vigant, F., Santos, N.C. & Lee, B. Broad-spectrum antivirals against viral fusion. Nat Rev Microbiol 13, 426-437 (2015).
Vigerust, D. J. & McCullers, J. A. Chloroquine is effective against influenza A virus in vitro but not in vivo. Influenza Other Respir Viruses 1, 189-192 (2007).
Vincent, M. J. et al. Chloroquine is a potent inhibitor of SARS coronavirus infection and spread. Virol J 2, 69 (2005).
Wang, M. et al. Remdesivir and chloroquine effectively inhibit the recently emerged novel coronavirus (2019-nCoV) in vitro. 30, 269-271 Cell Res (2020).
Wang, X. et al. The anti-influenza virus drug, arbidol is an efficient inhibitor of SARS-CoV-2 in vitro. Cell Discov 6, 28 (2020).
Woo, P. C. et al. Relative rates of non-pneumonic SARS coronavirus infection and SARS coronavirus pneumonia. Lancet 363, 841-845 (2004).
Xia, S. et al. Inhibition of SARS-CoV-2 (previously 2019-nCoV) infection by a highly potent pan-coronavirus fusion inhibitor targeting its spike protein that harbors a high capacity to mediate membrane fusion. Cell Res 30, 343-355 (2020).
Yan, N. & Chen, Z. J. Intrinsic antiviral immunity. Nat Immunol 13, 214-222 (2012)
Yeung, M. L. et al. MERS coronavirus induces apoptosis in kidney and lung by upregulating Smad7 and FGF2. Nat Microbiol 1, 16004 (2016).
Yu, Y. et al. A peptide-based viral inactivator inhibits Zika virus infection in pregnant mice and fetuses. Nat Commun 8, 15672 (2017).
Yuan, S. et al. SREBP-dependent lipidomic reprogramming as a broad-spectrum antiviral target. Nat Commun 10, 120 (2019).
Zhao, H. et al. A broad-spectrum virus- and host-targeting peptide against respiratory viruses including influenza virus and SARS-CoV-2. Nat Commun 11, 4252 (2020).
Zhao, H. et al. A novel peptide with potent and broad-spectrum antiviral activities against multiple respiratory viruses. Sci Rep 6, 22008 (2016).
Zhao, H. et al. Dual-functional peptide with defective interfering genes effectively protects mice against avian and seasonal influenza. Nat Commun 9, 2358 (2018).
Zhao, H. et al. Novel residues in the PA protein of avian influenza H7N7 virus affect virulence in mammalian hosts. Virology 498, 1-8 (2016).
Zheng, B. J. et al. Delayed antiviral plus immunomodulator treatment still reduces mortality in mice infected by high inoculum of influenza A/H5N1 virus. Proc Natl Acad Sci USA 105, 8091-8096 (2008).
Zhou, Y. et al. Protease inhibitors targeting coronavirus and filovirus entry. Antiviral Res 116, 76-84 (2015).
Zhu, Z. et al. Arbidol monotherapy is superior to lopinavir/ritonavir in treating COVID-19. J Infect 81, e21-e23 (2020).

1. An antiviral agent comprising a multivalent peptide, wherein the multivalent peptide comprises three or more copies of one or a combination of peptides selected from the group consisting of P9R (SEQ ID NO:2) and P9R-like peptides derived from P9R,
wherein at least three of the peptides that comprise the multivalent peptide branch from one or more of the peptides that comprise the multivalent peptide.

2. The antiviral agent of claim 1, wherein the multivalent peptide comprises six or more copies of the peptides,
wherein at least six of the peptides that comprise the multivalent peptide branch from one or more of the peptides that comprise the multivalent peptide.

3. The antiviral agent of claim 1, wherein the multivalent peptide comprises eight or more copies of the peptides,
wherein at least eight of the peptides that comprise the multivalent peptide branch from one or more of the peptides that comprise the multivalent peptide.

4. The antiviral agent of claim 1, wherein at least three of the peptides that branch from one or more of the peptides that comprise the multivalent peptide branch from a central point in the multivalent peptide.

5. The antiviral agent of claim 1, wherein at least six of the peptides that branch from one or more of the peptides that comprise the multivalent peptide branch from a central point in the multivalent peptide.

6. The antiviral agent of claim 1, wherein at least eight of the peptides that branch from one or more of the peptides that comprise the multivalent peptide branch from a central point in the multivalent peptide.

7. The antiviral agent of claim 1, wherein the peptides that branch from one or more of the peptides that comprise the multivalent peptide branch from a central point in the multivalent peptide.

8. The antiviral agent of claim 1, wherein the P9R-like peptides are characterized in that the P9R-like peptide inhibits endosomal acidification and retains virus binding as determined by an in vitro endosomal acidification,
optionally compared to a control, and/or a peptide-virus binding assay.

9. The antiviral agent of claim 1, wherein the peptides that comprise the multivalent peptide each consist of P9R (SEQ ID NO:2).

10. The antiviral agent of claim 1, wherein one or more of the peptides that comprise the multivalent peptide has a net positive charge of at least 5,
optionally wherein one or more of the peptides that comprise the multivalent peptide has a net positive charge of about 5.6.

11. The antiviral agent of claim 1, wherein the peptides that comprise the multivalent peptide each has a net positive charge of at least 5,
optionally wherein the peptides that comprise the multivalent peptide each has a net positive charge of about 5.6.

12-15. (canceled)

16. An antiviral agent comprising P9R (SEQ ID NO:2) or a P9R-like peptide derived from P9R.

17. The antiviral agent of claim 16, wherein the P9R-like peptide is characterized in that the P9R-like peptide inhibits endosomal acidification and retains virus binding as determined by an in vitro endosomal acidification,
optionally compared to a control, and/or a peptide-virus binding assay.

18. The antiviral agent of claim 16, consisting of P9R (SEQ ID NO:2).

19. The antiviral agent of claim 16, wherein the antiviral agent has a net positive charge of at least 5,
optionally wherein the antiviral agent has a net positive charge of about 5.6.

20-21. (canceled)

22. A composition comprising a therapeutically effective amount of the antiviral agent of claim 1 and a pharmaceutically acceptable carrier.

23. An antiviral composition comprising arbidol, chloroquine, and camostat.

24. The composition of claim 22, wherein the antiviral agent inhibits antiviral replication in the subject.

25. The composition of claim 22, wherein the composition is a unit dosage form.

26. The composition of claim 25, wherein the unit dosage form is a tablet, a capsule, or a form suitable for intranasal or pulmonary delivery.

27. (canceled)

28. The composition of claim 25, wherein the unit dosage form is an injectable form,
optionally wherein the composition further comprises a pharmaceutically acceptable carrier for injection to a human.

29. A method of treating a viral infection in a subject in need thereof, the method comprising
administering an effective amount of the composition of claim 22, to the subject.

30. The method of claim 29, wherein the infection is caused by a respiratory virus,
optionally wherein the virus is selected from the group consisting of zika virus, enterovirus-A7, ebola virus, influenza virus, SARS-CoV-2, SARS-CoV, MERS-CoV, the A(H1N1)pdm09 virus, avian influenza A(H7N9) virus, and the non-enveloped rhinovirus.

31. The method of claim 29, wherein the infection is caused by a pH-dependent virus that requires endosomal acidification for virus-host membrane fusion.

32. The method of claim 29, wherein the composition is administered parenterally orally, intranasally, or by pulmonary administration.

33-34. (canceled)


SEQUENCE LISTING (19 sequences)

SEQ ID NO: 1 - protein, 30 aa, Mus musculus
NGAICWGPCPTAFRQIGNCGKFKVRCCKIR

SEQ ID NO: 2 - protein, 30 aa, Mus musculus
NGAICWGPCPTAFRQIGNCGRFRVRCCRIR

SEQ ID NO: 3 - protein, 33 aa, Mus musculus
NGAICWGPCPTAFRQIGNCGHFKVRCCKIRDED

SEQ ID NO: 4 - protein, 30 aa, Mus musculus
NGAHSWHPNETHFRQIHNSGRHRVRSHRIR

SEQ ID NO: 5 - DNA, 30 bp, Artificial Sequence
  SARS-CoV-2 S-F
cctactaaattaaatgatctctgctttact

SEQ ID NO: 6 - DNA, 21 bp, Artificial Sequence
  SARS-CoV-2 S-R
caagctataacgcagcctgta

SEQ ID NO: 7 - DNA, 25 bp, Artificial Sequence
  MERS-CoV NP-F
caaaaccttccctaagaaggaaaag

SEQ ID NO: 8 - DNA, 22 bp, Artificial Sequence
  MERS-CoV NP-R
gctcctttggaggttcagacat

SEQ ID NO: 9 - DNA, 19 bp, Artificial Sequence
  SARS-CoV NP-F
accagaatggaggacgcaa

SEQ ID NO: 10 - DNA, 25 bp, Artificial Sequence
  SARS-CoV NP-R
gctgtgaaccaagacgcagtattat

SEQ ID NO: 11 - DNA, 21 bp, Artificial Sequence
  H1N1 M-F
cttctaaccgaggtcgaaacg

SEQ ID NO: 12 - DNA, 22 bp, Artificial Sequence
  H1N1 M-R
ggcattttggacaaakcgtcta

SEQ ID NO: 13 - DNA, 21 bp, Artificial Sequence
  H7N9 M-F
cttctaaccgaggtcgaaacg

SEQ ID NO: 14 - DNA, 22 bp, Artificial Sequence
  H7N9 M-R
ggcattttggacaaakcgtcta

SEQ ID NO: 15 - DNA, 16 bp, Artificial Sequence
  Rhinovirus 5′UTR-F
agccygcgtggckgcc

SEQ ID NO: 16 - DNA, 16 bp, Artificial Sequence
  Rhinovirus 5′UTR-R
agccygcgtggtgccc

SEQ ID NO: 17 - DNA, 22 bp, Artificial Sequence
  Probe
tccggcccctgaatgyggctaa

SEQ ID NO: 18 - DNA, 23 bp, Artificial Sequence
  parainfluenza virus 3 HPIV3_Inner-F
agctatyactagyatctcagggt

SEQ ID NO: 19 - DNA, 20 bp, Artificial Sequence
  parainfluenza virus 3 HPIV3_Inner-Rm
cccaatctgatccactgtgt